(19) United States
(12) Patent Application Publication
(10) Pub. No.: US 2025/0352809 A1
(43) Pub. Date: Nov. 20, 2025

(54) CROSSTALK PROTECTION FOR USE IN MULTI-CHAMBER LEADLESS PACEMAKER SYSTEMS

(71) Applicant: Pacesetter, Inc., Sylmar, CA (US)

(72) Inventors: Lac T. La, San Jose, CA (US); Matthew G. Fishler, Scotts Valley, CA (US); Weiqun Yang, Cupertino, CA (US)

(73) Assignee: Pacesetter, Inc., Sylmar, CA (US)

(21) Appl. No.: 19/282,924

(22) Filed: Jul. 28, 2025

Related U.S. Application Data
(63) Continuation of application No. PCT/US2024/051760, filed on Oct. 17, 2024.
(60) Provisional application No. 63/591,717, filed on Oct. 19, 2023.

(51) Int. Cl. A61N 1/375; A61N 1/368

(52) U.S. Cl. CPC A61N 1/3756; A61N 1/368; A61N 1/37512

(57) ABSTRACT

A dual chamber leadless pacemaker (LP) system includes a first leadless pacemaker (LP1) and a second leadless pacemaker (LP2), wherein the LP1 is configured to be implanted in or on a first cardiac chamber and to deliver pacing pulses to the first cardiac chamber, and the LP2 is configured to be implanted in or on a second cardiac chamber and to deliver pacing pulses to the second cardiac chamber. Information is obtained about a magnitude of the pacing pulses that the LP2 is configured to deliver to the second cardiac chamber and/or a sensitivity of a sense circuit of the LP1 that is configured to be used by the LP1 to detect intrinsic depolarizations of the first cardiac chamber. A crosstalk protection duration is determined based on at least some of the information so that when crosstalk protection is perform, it is performed for an appropriate duration.

### PRIORITY CLAIM

This application is a continuation of and claims priority to International Application No. PCT/US2024/051760, filed Oct. 17, 2024, which claims priority to U.S. Provisional Patent Application No. 63/591,717, filed Oct. 19, 2023. Priority is claimed to each of the above applications, and each of the above applications is incorporated by reference as if set forth fully herein in its entirety.

### FIELD OF TECHNOLOGY

Embodiments described herein generally relate to leadless pacemakers (LPs), multi-chamber leadless pacemaker (LP) systems, methods for use therewith, and non-implantable programmers for use therewith.

### BACKGROUND

Conventional pacemakers typically include a housing (aka “can”), which houses a controller (e.g., processor), and one or more intravascular leads that extend from the housing. Each of the leads includes one or more electrodes that can be used for sensing cardiac electrical activity and for delivering pacing stimulation. Such conventional pacemakers can support single-chamber operating modes (e.g., VVI, AAI) and dual-chamber operating modes (e.g., DDD, VDD), depending upon how many leads are used and how the conventional pacemakers are programmed.

Over the past few years, physicians have started implanting leadless pacemakers (LPs) in place of conventional pacemakers which require attached electrical leads. This is beneficial because such leads may fail and/or migrate more often than desired. Early LPs were implanted as standalone devices in a single cardiac chamber, e.g., the right ventricular (RV) chamber, and thus supported single-chamber operating modes (e.g., WVI). There is now a desire to start implanting and optionally coordinating multiple LPs in one or multiple cardiac chambers, e.g., in the RV chamber and in the right atrial (RA) chamber, to provide for a dual chamber LP system that is capable of supporting dual-chamber operating modes (e.g., DDD, VDD). More generally, there is a move towards implanting two LPs in (or on) two or more cardiac chambers to provide for multi-chamber pacing.

A conventional pacemaker includes one central processing unit (CPU) that manages cardiac activity in multiple cardiac chambers of a patient's heart using an individual lead for each cardiac chamber of a plurality of cardiac chambers (e.g., one lead for the right atrium, and another lead for the right ventricle). This allows the conventional pacemaker to sense intrinsic activity in multiple cardiac chambers and provide pacing support accordingly, as well as maintain atrioventricular (AV) synchrony. This conventional pacemaker configuration also has a distinct advantage of providing inherent protection against crosstalk, where “crosstalk” is when a pace pulse delivered by one lead and electrode combination is inappropriately detected as an intrinsic myocardial depolarization by a different lead and electrode combination (e.g., located in a different cardiac chamber of the heart). Since a conventional pacemaker provides the pace stimulus, the conventional pacemaker can also coordinate when and for how long to blank or ignore the sensed signals from all other leads and electrode combinations, thus mitigating the risk of crosstalk adversely affecting the operation of the conventional pacemaker. Leadless pacemakers (LPs), however, do not have this inherent common knowledge, since each LP that is implanted in (or on) a different cardiac chamber is an independent device with its own controller (e.g., a CPU). Coordinated LP systems therefore rely on some form of wireless communication to synchronize with one another LP and otherwise exchange information.

### SUMMARY

Certain embodiments of the present technology are directed to a system for use with or including a first leadless pacemaker (LP1) and a second leadless pacemaker (LP2), wherein the LP1 is configured to be implanted in or on a first cardiac chamber of a patient's heart and to deliver pacing pulses to the first cardiac chamber, and the LP2 is configured to be implanted in or on a second cardiac chamber of the patient's heart and to deliver pacing pulses to the second cardiac chamber. The system comprises a controller configured to obtain information about one or more: a magnitude of the pacing pulses that the LP2 is configured to deliver to the second cardiac chamber; or a sensitivity of a sense circuit of the LP1 that is configured to be used by the LP1 to detect intrinsic depolarizations of the first cardiac chamber. The controller is also configured to determine a crosstalk protection duration based on at least some of the information, wherein the crosstalk protection duration after being determined is used by the LP1 to perform crosstalk protection for the crosstalk protection duration in response to the LP1 detecting possible crosstalk that may be caused by the LP2 delivering one of the pacing pulses.

In accordance with certain embodiments, the controller is configured to obtain information about the magnitude of the pacing pulses that the LP2 is configured to deliver to the second cardiac chamber; and determine the crosstalk protection duration based on the magnitude of the pacing pulses such that there is a positive correlation between the magnitude of the pacing pulses and the crosstalk protection duration. In an embodiment, where the controller is configured to determine the crosstalk protection duration based on the magnitude of the pacing pulses, such that there is a positive correlation between the magnitude of the pacing pulses and the crosstalk protection duration, the controller is configured to determine that the crosstalk duration has a first duration if the pacing pulses have a first magnitude and have a second duration that is longer than the first duration if the pacing pulses has a second magnitude that is greater than the first magnitude. The controller can also be configured to determine that the crosstalk duration has a third duration that is shorter than the first duration if the pacing pulses have a third magnitude that is less than the first magnitude.

In accordance with certain embodiments, the controller is configured to obtain information about the magnitude of the pacing pulses that the LP2 is configured to deliver to the second cardiac chamber by obtaining information about at least one of a pulse amplitude or a pulse width of the pacing pulses that the LP2 is configured to deliver to the second cardiac chamber.

In accordance with certain embodiments, the controller is configured to obtain information about the sensitivity of the sense circuit of the LP1. In certain such embodiments, the sensitivity of the sense circuit of the LP1 is specified by a sense detection threshold of the sense circuit of the LP1 that is configured to be used by the LP1 to detect intrinsic depolarizations of the first cardiac chamber, and the controller is configured to determine the crosstalk protection duration based on the sense detection threshold of the sense circuit such that there is a negative correlation between the sense detection threshold of the sense circuit and the crosstalk protection duration. In an embodiment, where the controller is configured to determine the crosstalk protection duration based on the sense detection threshold of the sense circuit of the LP1, such that there is a negative correlation between the sense detection threshold of the sense circuit of the LP1 and the crosstalk protection duration, the controller is configured to determine that the crosstalk duration has a first duration if the sense detection threshold of the sense circuit of the LP1 has a first magnitude and has a second duration that is shorter than the first duration if the sense detection threshold of the sense circuit of the LP1 has a second magnitude that is greater than the first magnitude. Such a controller can also be configured to determine that the crosstalk duration has a third duration that is longer than the first duration if the sense detection threshold of the sense circuit of the LP1 has a third magnitude that is less than the first magnitude. Alternatively, the sensitivity of the sense circuit of the LP1 is specified by a gain of the sense circuit of the LP1 that is configured to be used by the LP1 to detect intrinsic depolarizations of the first cardiac chamber, and the controller is configured to determine the crosstalk protection duration based on the gain of the sense circuit such that there is a positive correlation between the gain of the sense circuit and the crosstalk protection duration. In an embodiment, where the controller is configured to determine the crosstalk protection duration based on the gain of the sense circuit of the LP1 (that is configured to be used to detect intrinsic depolarizations of the first cardiac chamber), such that there is a positive correlation between the gain of the sense circuit (that is configured to be used to detect intrinsic depolarizations of the first cardiac chamber) and the crosstalk protection duration, the controller is configured to determine that the crosstalk protection duration has a first duration if the gain circuit has a first gain and has a second duration that is longer than the first duration if the gain circuit has a second gain that is greater than the first gain. Such a controller can also be configured to determine that the crosstalk duration has a third duration that is shorter than the first duration, if the gain circuit has a third gain that is less than the first gain.

In accordance with certain embodiments, the controller is configured to obtain information about the magnitude of the pacing pulses that the LP2 is configured to deliver to the second cardiac chamber, and obtain information about the sensitivity of the sense circuit of the LP1 that is configured to be used by the LP1 to detect intrinsic depolarizations of the first cardiac chamber; and determine the crosstalk protection duration based on the magnitude of the pacing pulses and the sensitivity of the sense circuit.

In accordance with certain embodiments, the controller is configured to determine the crosstalk protection duration also based on a scaling factor that is related to at least one of a distance between the LP1 and the LP2 or an angle of the LP1 and the LP2 relative to one another. In certain such embodiments, the controller is configured to: determine a preliminary crosstalk protection duration based on at least some of the information; determine a scaling factor based on a distance between the LP1 and the LP2 or an angle of the LP1 and the LP2 relative to one another; and determine the cross talk protection duration by scaling the preliminary crosstalk protection duration by the scaling factor.

In accordance with certain embodiments, the system comprises a portion of the LP1 that includes the controller, wherein the controller of the LP1 is configured to monitor for and detect possible crosstalk that may be caused by the LP2 delivering one of the pacing pulses; and initiate performance of crosstalk protection for the crosstalk protection duration in response to detecting possible crosstalk that may be caused by the LP2 delivering one of the pacing pulses.

In accordance with certain embodiments, the controller of the LP1 is configured to: dynamically adjust the sensitivity of the sense circuit of the LP1; and determine the crosstalk protection duration based on the sensitivity such that the crosstalk protection duration is updated by the controller of the LP1 when the controller of the LP1 adjusts the sensitivity of the sense circuit of the LP1.

In accordance with certain embodiments, a controller of the LP2 is configured to dynamically adjust the magnitude of the pacing pulses that the LP2 is configured to deliver to the second cardiac chamber and to inform the controller of LP1 of adjustments made to the magnitude of the pacing pulses; and the controller of the LP1 is configured to determine the crosstalk protection duration based on the magnitude of the pacing pulses that the LP2 is configured to deliver to the second cardiac chamber such that there is a positive correlation between the magnitude of the pacing pulses and the crosstalk protection duration and such that the crosstalk protection duration is updated by the controller of the LP1 in response to the controller of the LP1 being informed of the adjustments made to the magnitude of the pacing pulses.

In accordance with certain embodiments, the system comprises a non-implantable programmer that includes the controller (that is configured to determine the crosstalk protection duration) and is configured to communicate with the LP1 and the LP2 (directly or through an intermediary such as another IMD), wherein the non-implantable programmer is configured to program the crosstalk protection duration into a memory or one or more registers of the LP1 so that the crosstalk protection duration is available for use by the LP1 when possible crosstalk that may be caused by the LP2 delivering one of the pacing pulses is detected by the LP1.

In accordance with certain embodiments, the system comprises a portion of the LP1 that includes the controller, wherein the controller of the LP1 is configured to provide crosstalk protection for the crosstalk protection duration by causing at least one of: blanking of the sense circuit of the LP1 for the crosstalk protection duration; ignoring of any possible intrinsic depolarization detected using the sense circuit of the LP1 during the crosstalk protection duration; disabling of the sense circuit of the LP1 for the crosstalk protection duration; ignoring of any interrupts produced in response to the sense circuit being used to detect a possible intrinsic depolarization during the crosstalk protection duration; and disabling of generating of interrupts that may be produced in response to the sense circuit detecting an intrinsic depolarization during the crosstalk protection duration. More generally, during a crosstalk protection duration associated with the LP1, the LP1 (and more specifically, the controller thereof) is prevented from detecting, and/or ignores detections of, any possible intrinsic depolarization(s).

In accordance with certain embodiments, the system comprises a portion of the LP1 that includes the controller, and the controller of the LP1 is configured to determine whether the possible crosstalk that was detected is part of a valid message transmitted by another device; and terminate the crosstalk protection for a remainder of the crosstalk protection duration in response to determining that the possible crosstalk that was detected is part of the valid message transmitted by another device.

Certain embodiments of the present technology are directed to a leadless pacemaker configured to communicate with another leadless pacemaker, wherein the leadless pacemaker is configured to be implanted in or on a first cardiac chamber of a patient's heart and to deliver pacing pulses to the first cardiac chamber, the other leadless pacemaker is configured to be implanted in or on a second cardiac chamber of the patient's heart and to deliver pacing pulses to the second cardiac chamber. The leadless pacemaker includes a sense circuit and a controller. The sense circuit is configured to be used to detect intrinsic depolarizations of the first cardiac chamber. The controller is configured to obtain information about one or more of the following a magnitude of the pacing pulses that the other leadless pacemaker is configured to deliver to the second cardiac chamber, or a sensitivity of the sense circuit. The controller is also configured to: determine a crosstalk protection duration based on at least some of the information; monitor for possible crosstalk that may be caused by the other leadless pacemaker delivering one of the pacing pulses; and perform crosstalk protection for the crosstalk protection duration in response to detecting the possible crosstalk that may be caused by the other leadless pacemaker delivering one of the pacing pulses.

In accordance with certain embodiments, the controller is configured to obtain information about the magnitude of the pacing pulses that the other leadless pacemaker is configured to deliver to the second cardiac chamber, and based thereon determine the crosstalk protection duration such that there is a positive correlation between the magnitude of the pacing pulses and the crosstalk protection duration.

In accordance with certain embodiments, the controller is configured to obtain information about a sense detection threshold of the sense circuit and determine the crosstalk protection duration based on the sense detection threshold of the sense circuit such that there is a negative correlation between the sense detection threshold of the sense circuit and the crosstalk protection duration; or obtain information about a gain of the sense circuit and determine the crosstalk protection duration based on the gain of the sense circuit such that there is a positive correlation between the gain of the sense circuit and the crosstalk protection duration.

In accordance with certain embodiments, the controller is configured to obtain information about the magnitude of the pacing pulses that the other leadless pacemaker is configured to deliver to the second cardiac chamber; obtain information about the sensitivity of the sense circuit; and determine the crosstalk protection duration based on the magnitude of the pacing pulses and the sensitivity of the sense circuit.

In accordance with certain embodiments, the controller is configured to determine the crosstalk protection duration also based on a scaling factor that is related to at least one of a distance between the leadless pacemaker and the other leadless pacemaker or an angle of the leadless pacemaker and the other leadless pacemaker relative to one another. In certain such embodiments, the controller is configured to: determine a preliminary crosstalk protection duration based on at least some of the information; determine a scaling factor based on a distance between the LP1 and the LP2 or an angle of the LP1 and the LP2 relative to one another; and determine the cross talk protection duration by scaling the preliminary crosstalk protection duration by the scaling factor.

In accordance with certain embodiments, the controller is configured to dynamically adjust the sensitivity of the sense circuit; and determine the crosstalk protection duration based on the sensitivity of the sense circuit, such that the crosstalk protection duration is updated when the sensitivity of the sense circuit is adjusted.

In accordance with certain embodiments, the controller is configured to determine the crosstalk protection duration based on the magnitude of the pacing pulses that the other leadless pacemaker is configured to deliver to the second cardiac chamber, such that there is a positive correlation between the magnitude of the pacing pulses and the crosstalk protection duration and such that the crosstalk protection duration is updated in response to being informed of the adjustments made to the magnitude of the pacing pulses that the other leadless pacemaker is configured to deliver to the second cardiac chamber.

In accordance with certain embodiments, the controller is configured to provide crosstalk protection, for the crosstalk protection duration by causing at least one of the following: blanking of the sense circuit for the crosstalk protection duration; ignoring of any possible intrinsic depolarization detected using the sense circuit during the crosstalk protection duration; disabling of the sense circuit for the crosstalk protection duration; ignoring of any interrupts produced in response to the sense circuit being used to detect a possible intrinsic depolarization during the crosstalk protection duration; or disabling of generating of interrupts that may be produced in response to the sense circuit detecting an intrinsic depolarization during the crosstalk protection duration. More generally, during a crosstalk protection duration associated with an LP, the LP (and more specifically, the controller thereof) is prevented from detecting, and/or ignores detections of, any possible intrinsic depolarization(s).

In accordance with certain embodiments, the controller is configured to determine whether the possible crosstalk that was detected is part of a valid message transmitted by another device; and terminate the crosstalk protection for a remainder of the crosstalk protection duration, in response to determining that the possible crosstalk that was detected is part of the valid message transmitted by another device.

Certain embodiments of the present technology are directed to a crosstalk protection method for use with a dual chamber leadless pacemaker (LP) system including a first leadless pacemaker (LP1) and a second leadless pacemaker (LP2), wherein the LP1 is configured to be implanted in or on a first cardiac chamber of a patient's heart and to deliver pacing pulses to the first cardiac chamber, and the LP2 is configured to be implanted in or on a second cardiac chamber of the patient's heart and to deliver pacing pulses to the second cardiac chamber. The method comprises obtaining information about one or more of the following: a magnitude of the pacing pulses that the LP2 is configured to deliver to the second cardiac chamber; or a sensitivity of a sense circuit of the LP1 that is configured to be used by the LP1 to detect intrinsic depolarizations of the first cardiac chamber. The method also includes determining a crosstalk protection duration based on at least some of the information. The method additionally includes, the LP1 monitoring for and detecting possible crosstalk that may by caused by the LP2 delivering one of the pacing pulses; and the LP1, in response to detecting the possible crosstalk that may by caused by the LP2 delivering one of the pacing pulses, initiating providing crosstalk protection for the crosstalk protection duration.

In accordance with certain embodiments, the obtaining information comprises obtaining information about the magnitude of the pacing pulses that the LP2 is configured to deliver to the second cardiac chamber; and the determining the crosstalk protection duration is based on the magnitude of the pacing pulses such that there is a positive correlation between the magnitude of the pacing pulses and the crosstalk protection duration. In accordance with certain embodiments, the obtaining information about the magnitude of the pacing pulses that the LP2 is configured to deliver to the second cardiac chamber, comprises obtaining information about at least one of a pulse amplitude or a pulse width of the pacing pulses that the LP2 is configured to deliver to the second cardiac chamber.

In accordance with certain embodiments, the obtaining information comprises obtaining information about the sensitivity of the sense circuit of the LP1. In certain such embodiments, the sensitivity of the sense circuit of the LP1 is specified by a sense detection threshold of the sense circuit of the LP1 that is configured to be used by the LP1 to detect intrinsic depolarizations of the first cardiac chamber, and the determining the crosstalk protection duration is based on the sense detection threshold of the sense circuit such that there is a negative correlation between the sense detection threshold of the sense circuit and the crosstalk protection duration. Alternatively, the sensitivity of the sense circuit of the LP1 is specified by a gain of the sense circuit of the LP1 that is configured to be used by the LP1 to detect intrinsic depolarizations of the first cardiac chamber, and the determining the crosstalk protection duration is based on the gain of the sense circuit such that there is a positive correlation between the gain of the sense circuit and the crosstalk protection duration.

In accordance with certain embodiments, the obtaining information comprises obtaining information about the magnitude of the pacing pulses that the LP2 is configured to deliver to the second cardiac chamber, and obtaining information about the sensitivity of the sense circuit of the LP1 that is configured to be used by the LP1 to detect intrinsic depolarizations of the first cardiac chamber; and the determining the crosstalk protection duration is based on the magnitude of the pacing pulses and the sensitivity of the sense circuit.

In accordance with certain embodiments, the determining the crosstalk protection duration is also based on a scaling factor that is related to at least one of a distance between the LP1 and the LP2 or an angle of the LP1 and the LP2 relative to one another. In certain such embodiments, the determining the crosstalk protection duration is also based on a scaling factor comprises: determining a preliminary crosstalk protection duration based on at least some of the information; determining a scaling factor based on a distance between the LP1 and the LP2 or an angle of the LP1 and the LP2 relative to one another; and determining the cross talk protection duration by scaling the preliminary crosstalk protection duration by the scaling factor.

In accordance with certain embodiments, the determining the crosstalk protection duration is performed by a controller of the LP1.

In accordance with certain embodiments, the controller of the LP1 is configured to dynamically adjust the sensitivity of the sense circuit of the LP1; and the determining the crosstalk protection duration is based on the sensitivity such that crosstalk protection duration is updated by the controller of the LP1 when the controller of the LP1 adjusts the sensitivity of the sense circuit of the LP1.

In accordance with certain embodiments, a controller of the LP2 is configured to dynamically adjust the magnitude of the pacing pulses that the LP2 is configured to deliver to the second cardiac chamber, and to inform the controller of LP1 of adjustments made to the magnitude of the pacing pulses; and the determining the crosstalk protection duration is based on the magnitude of the pacing pulses that the LP2 is configured to deliver to the second cardiac chamber such that there is a positive correlation between the magnitude of the pacing pulses and the crosstalk protection duration and such that the crosstalk protection duration is updated by the controller of the LP1 in response to the controller of the LP1 being informed by the LP2 of the adjustments made to the magnitude of the pacing pulses.

In accordance with certain embodiments, the obtaining the information and the determining the crosstalk protection duration are performed by a non-implantable programmer; and the method further comprises the non-implantable programmer programming the crosstalk protection duration into a memory or one or more registers of the LP1, so that the crosstalk protection duration is available for use by the LP1 when possible crosstalk (that may be caused by the LP2 delivering one of the pacing pulses) is detected by the LP1.

In accordance with certain embodiments, the providing crosstalk protection for the crosstalk protection duration comprises at least one of the following: blanking the sense circuit of the LP1 for the crosstalk protection duration; ignoring any possible intrinsic depolarization detected using the sense circuit of the LP1 during the crosstalk protection duration; disabling the sense circuit of the LP1 for the crosstalk protection duration; ignoring any interrupts produced in response to the sense circuit being used to detect a possible intrinsic depolarization during the crosstalk protection duration; or disabling generating of interrupts that may be produced in response to the sense circuit detecting an intrinsic depolarization during the crosstalk protection duration. More generally, during a crosstalk protection duration, the possible intrinsic depolarizations are prevented from detecting, and/or ignored.

In accordance with certain embodiments, the method further comprises determining that the possible crosstalk that was detected is part of a valid message transmitted by another device, and in response thereto terminating the crosstalk protection for a remainder of the crosstalk protection duration.

This summary is not intended to be a complete description of the embodiments of the present technology. Other features and advantages of the embodiments of the present technology will appear from the following description in which the preferred embodiments have been set forth in detail, in conjunction with the accompanying drawings and claims.

### BRIEF DESCRIPTION OF THE DRAWINGS

Embodiments of the present technology relating to both structure and method of operation may best be understood by referring to the following description and accompanying drawings, in which similar reference characters denote similar elements throughout the several views:

FIG. 1 illustrates a system formed in accordance with certain embodiments herein as implanted in a heart.

FIG. 2 is a block diagram of a single LP in accordance with certain embodiments herein.

FIG. 3 illustrates an LP in accordance with certain embodiments herein.

FIG. 4 is a timing diagram demonstrating one embodiment of i2i communication for a paced event.

FIG. 5 is a timing diagram demonstrating one embodiment of i2i communication for a sensed event.

FIG. 6 is a timing diagram that is used to illustrate how a pacing pulse delivered by a second LP implanted in (or on) a remote cardia chamber may cause crosstalk that may be falsely detected as an intrinsic depolarization of a local cardiac chamber by a first LP implanted in (or on) the local cardiac chamber.

FIGS. 7A and 7B are high level flow diagrams that are used to summarize methods according to certain embodiments of the present technology.

FIG. 8 shows a block diagram of one embodiment of an IMD (e.g., LP) that is implanted into a patient as part of the implantable cardiac system in accordance with certain embodiments herein.

FIG. 9 shows a block diagram of one embodiment of an external device 109 for use in communicating with and/or programming the LPs and/or the ICD introduced in FIG. 1, or some of the type of implantable medical device (IMD), and which can be used to implement certain embodiments of the present technology.

### DETAILED DESCRIPTION

As noted above, there is now a desire to implant LPs in two or more cardiac chambers, e.g., in the RV chamber and in the RA chamber, to provide for a multi-chamber (e.g., dual chamber) LP system that is capable of supporting dual-chamber operating modes (e.g., DDD, VDD), or more generally, multi-chamber operation. An LP that is implanted in (or on), or configured to be implanted in (or on), the RV chamber may be referred to herein as a ventricular LP, or a vLP. An LP that is implanted in (or on), or configured to be implanted in (or on), the RA chamber may be referred to herein as an atrial LP, or an aLP.

When there is more than one LP implanted in and/or on a patient's heart, there is a risk that a pacing pulse delivered by one LP may be detected by a sense circuit of another LP and incorrectly interpreted as an intrinsic activation event. In other words, there is a risk that an LP implanted in or on a first cardiac chamber may detect and interpret crosstalk, caused by another LP implanted in or on a second cardiac chamber pacing the second cardiac chamber, as an intrinsic event (aka an intrinsic depolarization) of the first cardiac chamber.

When implant-to-implant (i2i) communication between the LPs is enabled and functioning properly, an LP may transmit an i2i message (and more specifically, a pacing event i2i message) immediately before delivering a pacing pulse, in which case the i2i message alerts the other LP of the impending pacing pulse, so that the other LP can preemptively blank its sense circuit and prevent inappropriate crosstalk detection. However, when i2i communication in a multi-chamber LP system is unsuccessful, impeded, or disabled, there is a real risk that crosstalk (caused by one of the LPs delivering a pacing pulse) is detected (by another one of the LPs) and adversely affects the operation of the multi-chamber LP system. For example, where crosstalk is inadvertently interpreted as a sensed intrinsic cardiac event (aka a sensed intrinsic depolarization), pacing of a cardiac chamber may be inhibited when it actually should have been delivered, and/or the timing of one or more further pacing pulses may be adversely affected.

To mitigate against (and preferably prevent) crosstalk adversely affecting a dual chamber LP system (or more generally, a multi-chamber LP system), each LP of the system can independently monitor for possible crosstalk that may be caused by another LP delivering a pacing pulse. Then, in response to detecting possible crosstalk, the LP that detects the possible crosstalk can perform crosstalk protection for some period of time (aka duration), wherein the crosstalk protection can involve, for example, blanking a sense circuit for the period of time, which sense circuit the LP uses to monitor for intrinsic events of the chamber in (or on) which the LP is implanted. Alternatively, or additionally, the crosstalk protection (that is performed in response to detecting possible crosstalk) can involve ignoring any possible intrinsic depolarization detected using the sense circuit of the LP during the period of time, disabling the sense circuit of the LP for the period of time, ignoring any interrupts (produced in response to the sense circuit being used to detect a possible intrinsic depolarization) during the period of time, or disabling generating of interrupts (that may be produced in response to the sense circuit detecting an intrinsic depolarization) during the period of time, but is not limited thereto. Such crosstalk protection may be performed under the assumption that any possible intrinsic depolarization(s) that was/were detected while crosstalk protection is being performed was/were actually due to the crosstalk. More generally, during a crosstalk protection duration, any possible intrinsic depolarization(s) are prevented from detecting, and/or is/are ignored.

Various techniques can be used by an LP to detect possible crosstalk that may be caused by another LP, some of which techniques are described below. However, detecting possible crosstalk does not in itself mitigate against potential adverse effects of the possible crosstalk, since it would also be beneficial to determine a duration for crosstalk protection. Explained another way, while detecting possible crosstalk is beneficial, it would also be beneficial to determine a crosstalk protection duration, wherein the crosstalk protection duration can correspond to how long a sense circuit (used to sense for local intrinsic depolarizations) should be blanked in response to detecting possible crosstalk, or more generally how long crosstalk protection should be performed in response to detecting possible crosstalk.

Determining an appropriate crosstalk protection duration for use in a dual chamber LP system (and more generally, a multi-chamber LP system) is not a trivial endeavor, as there are various factors of an LP system that may affect the appropriate crosstalk protection duration. If a crosstalk protection duration is set too short, that may lead to a residual detection of crosstalk that may be falsely detected as an intrinsic depolarization. Conversely, if a crosstalk protection duration is set too long, that may lead to true local intrinsic events being missed (i.e., failing to be detected).

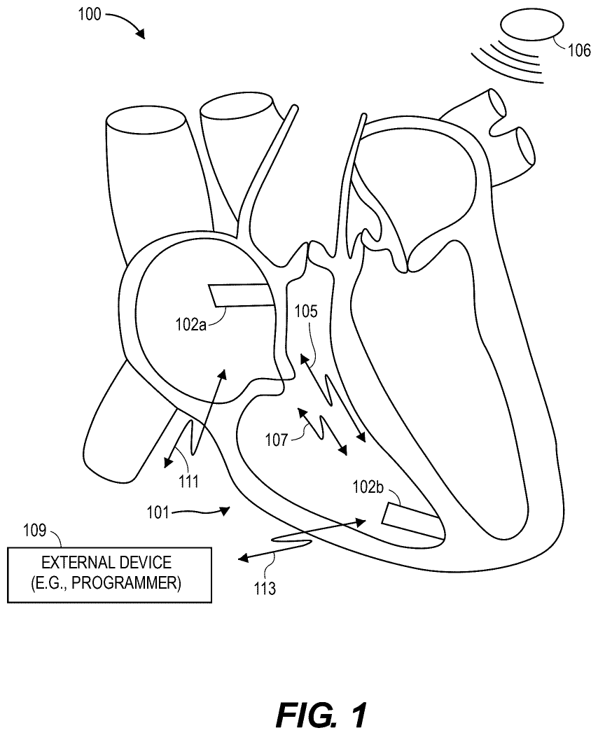

Certain embodiments of the present technology relate to systems, subsystems, and methods that specify and utilize an appropriate crosstalk protection duration for an LP of a multi-chamber LP system. FIGS. 1-5 describe an example dual chamber LP system, which optionally also includes a non-vascular ICD (NV-ICD), such as a subcutaneous-ICD (S-ICD), and an external device, such as a programmer.

FIG. 1 illustrates a system 100 that includes LPs 102a and 102b located in different chambers of a heart 101. LP 102a is located in a right atrium, and thus can also be referred to herein as an atrial LP (aLP). The LP 102b is located in a right ventricle, and thus can also be referred to herein as a ventricular LP (vLP). The aLP 102a and the vLP 102b can be referred to collectively herein as the LPs 102, or individually as an LP 102. Accordingly, when generally referring to an LP 102, the LP 102 can be the LP 102a or the LP 102b. The LPs 102a and 102b can communicate with one another to inform one another of various local physiologic activities, such as local intrinsic events, local paced events and the like. The LPs 102a and 102b may be constructed in a similar manner, but operate differently based upon which chamber the LP 102a or 102b is located.

In certain embodiments, LPs 102a and 102b communicate with one another, and/or with an ICD 106, by conductive communication through the same electrodes that are used for sensing and/or delivery of pacing therapy. The LPs 102a and 102b may also be able to use conductive communication to communicate with an external device, e.g., a programmer 109, having electrodes placed on the skin of a patient within with the LPs 102a and 102b are implanted. The LPs 102a and 102b can each alternatively, or additionally, include an antenna that would enable them to communicate with one another, the ICD 106 and/or an external device 109, using RF communication. Alternatively, or additionally, it is possible that the LPs 102a, 102b utilize another type of communication, such as inductive communication, in which case the LPs 102a, 102b can each include a respective inductive communication coil. Alternatively, or additionally, the LPs 102a, 102b could use conductive communication when communicating with each other and another type of communication, such as RF communication or inductive communication, when communicating the external device 109, such as programmer. While only two LPs are shown in FIG. 1, it is possible that more than two LPs can be implanted in a patient. For example, to provide for bi-ventricular pacing and/or cardiac resynchronization therapy (CRT), in addition to having LPs implanted in the right atrial (RA) chamber and the right ventricular (RV) chamber, a further LP can be implanted in the left ventricular (LV) chamber.

Each LP 102 uses two or more electrodes located within, on, or within a few centimeters of the housing of the LP, for pacing and sensing at the cardiac chamber. Where the LPs 102 communication using conductive communication, the electrodes of the LPs 102 can also be used for bidirectional conductive communication with one another, as well as with the programmer 109, and the ICD 106. It is noted that the term conductive communication and the term conducted communication are used interchangeably herein.

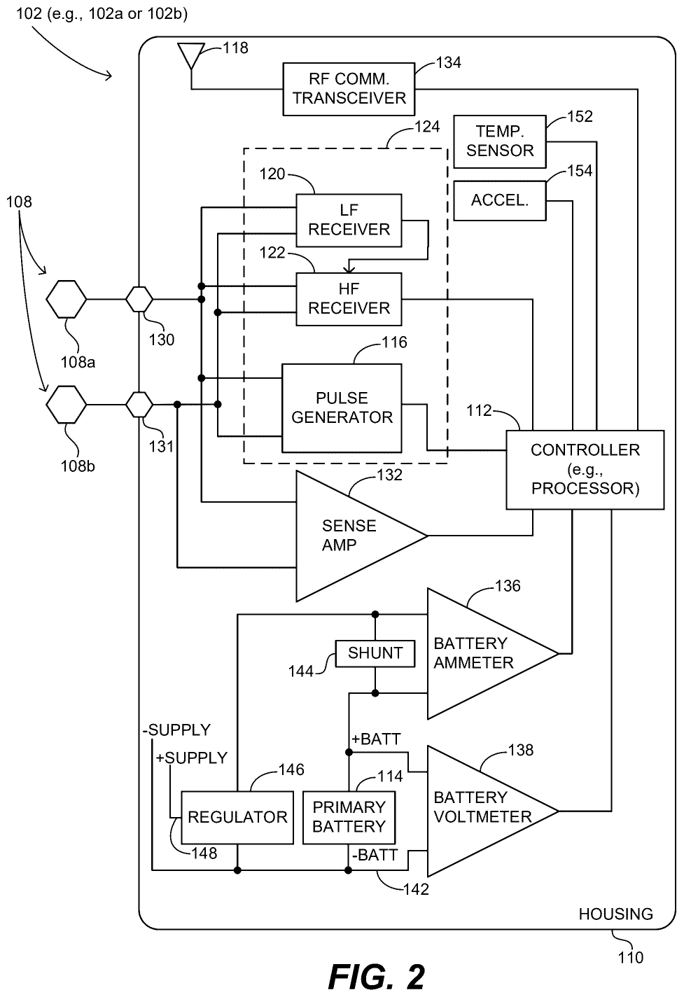

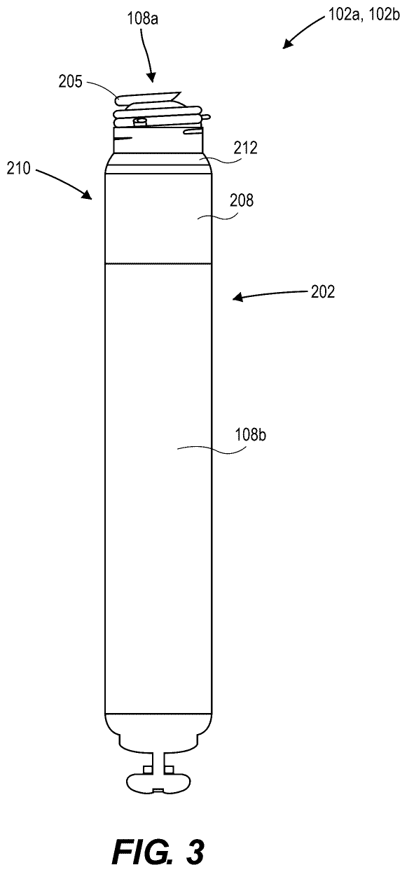

Referring to FIG. 2, a block diagram shows an embodiment for portions of the electronics within LPs 102a, 102b configured to provide conductive communication through the sensing/pacing electrode. One or more of LPs 102a and 102b include at least two leadless electrodes configured for delivering cardiac pacing pulses, sensing evoked and/or natural cardiac electrical signals, and uni-directional or bi-directional conductive communication. In FIG. 2 (and FIG. 3) the two electrodes shown therein are labeled 108a and 108b. Such electrodes can be referred to collectively as the electrodes 108, or individually as an electrode 108. An LP 102, or other type of IMD, can include more than two electrodes 108, depending upon implementation.

In FIG. 2, each of the LPs 102a, 102b is shown as including first and second receivers 120 and 122 that collectively define separate first and second conductive communication channels 105 and 107 (FIG. 1), (among other things) between LPs 102a and 102b. Although first and second receivers 120 and 122 are depicted, in other embodiments, each LP 102a, 102b may only include the first receiver 120, or more generally may include only a single receiver that is configured to receive conductive communication signals. It is also possible that an LP 102 may include additional receivers other than first and second receivers 120 and 122. As will be described in additional detail below, the pulse generator 116 can function as a transmitter that transmits i2i communication signals using the electrodes 108. In certain embodiments, LPs 102a and 102b may communicate over more than just first and second conductive communication channels 105 and 107. In certain embodiments, LPs 102a and 102b may communicate over one common communication channel 105. More specifically, LPs 102a and 102b can communicate conductively over a common physical channel via the same electrodes 108 that are also used to deliver pacing pulses.

The receivers 120 and 122 can also be referred to, respectively, as a low frequency (LF) receiver 120 and a high frequency (HF) receiver 122, because the receiver 120 is configured to monitor for one or more signals within a relatively low frequency range (e.g., below 100 kHz) and the receiver 122 is configured to monitor for one or more signals within a relatively high frequency range (e.g., above 100 kHz). In certain embodiments, the receiver 120 (and more specifically, at least a portion thereof) is always enabled and monitoring for a wakeup notice, which can simply be a wakeup pulse, within a specific low frequency range (e.g., between 1 kHz and 100 kHz); and the receiver 122 is selectively enabled by the receiver 120. The receiver 120 is configured to consume less power than the receiver 122 when both the first and second receivers are enabled. Accordingly, the receiver 120 can also be referred to as a low-power receiver 120, and the receiver 122 can also be referred to as a high-power receiver 122.

In accordance with certain embodiments, the low-power receiver 120 is incapable of receiving signals within the relatively high frequency range (e.g., above 100 kHz), but consumes significantly less power than the high-power receiver 122. This way the low-power receiver 120 is capable of always monitoring for a wakeup notice without significantly depleting the battery (e.g., 114) of the LP. In accordance with certain embodiments, the high-power receiver 122 is selectively enabled by the low-power receiver 120, in response to the low-power receiver 120 receiving a wakeup notice, so that the high-power receiver 122 can receive the higher frequency signals, and thereby handle higher data throughput needed for effective i2i communication without unnecessarily and rapidly depleting the battery of the LP (which the high-power receiver 122 may do if it were always enabled).

Since the receivers 120, 122 are used to receive conductive communication messages, the receivers 120, 122 can also be referred to as conductive communication receivers. In certain embodiments, each of the LPs 102 includes only a single conductive communication receiver.

In accordance with certain embodiments, when one of the LPs 102a and 102b senses an intrinsic event or delivers a paced event, the corresponding LP 102a, 102b transmits an implant event message to the other LP 102a, 102b. For example, when an aLP 102a senses/paces an atrial event, the aLP 102a transmits an implant event message including an event marker indicative of a nature of the event (e.g., intrinsic/sensed atrial event, paced atrial event). When a vLP 102b senses/paces a ventricular event, the vLP 102b transmits an implant event message including an event marker indicative of a nature of the event (e.g., intrinsic/sensed ventricular event, paced ventricular event). In certain embodiments, each LP 102a, 102b transmits an implant event message to the other LP 102a, 102b preceding the actual pace pulse so that the remote LP can blank its sense inputs in anticipation of that remote pace pulse (to prevent inappropriate crosstalk sensing). The above describe implant event messages are examples of i2i messages.

The implant event messages may be formatted in various manners. As one example, each event message may include a leading trigger pulse (also referred to as an LP wakeup notice, wakeup pulse, or wakeup signal) followed by an event marker. The notice trigger pulse (also referred to as the wakeup notice, wakeup pulse or wakeup signal) is transmitted over a first channel (e.g., with a pulse duration of approximately 10 us to approximately 1 ms and/or within a fundamental frequency range of approximately 1 kHz to approximately 100 kHz). The notice trigger pulse indicates that an event marker is about to be transmitted over a second channel (e.g., within a higher frequency range). The event marker can then be transmitted over the second channel.

The event markers may include data indicative of one or more events (e.g., a sensed intrinsic atrial activation for an aLP, a sensed intrinsic ventricular activation for a vLP). The event markers may include different markers for intrinsic and paced events. The event markers may also indicate start or end times for timers (e.g., an AV interval, a blanking interval, etc.). Optionally, the implant event message may include a message segment that includes additional/secondary information.

Optionally, the LP (or other IMD, such as an implantable cardiac monitor (ICM), subcutaneous ICD (SICD) or non-vascular ICD (NV-ICD)) that receives any i2i communication message from another LP (or other IMD) or from an external device may transmit a receive acknowledgement (ACK) indicating that the receiving LP (or other IMD) received the i2i communication message. In certain embodiments, where an LP (or other IMD) expects to receive an i2i communication message within a window, and fails to receive the i2i communication message within the window, the LP (or other IMD) may transmit a failure-to-receive acknowledgement indicating that the receiving LP (or other IMD) failed to receive the i2i communication message. The failure-to-receive acknowledgement message can also be referred to as a negative acknowledgement (NACK) message. An LP can receive a message from another LP that includes an indicator (e.g., an error code) in its payload, or header, that indicates to the LP that the other LP failed to receive an expected message from the LP. Other variations are also possible and within the scope of the embodiments described herein.

The event messages enable the LPs 102a, 102b to deliver synchronized therapy and additional supportive features (e.g., measurements, etc.). To maintain synchronous therapy, each of the LPs 102a and 102b is made aware (through the event messages) when an event occurs in the chamber containing the other LP 102a, 102b. Some embodiments described herein provide efficient and reliable processes to maintain synchronization between LPs 102a and 102b without maintaining continuous communication between LPs 102a and 102b. In accordance with certain embodiments herein, low power event messages/signaling may be maintained between LPs 102a and 102b synchronously or asynchronously.

For synchronous event signaling, LPs 102a and 102b may maintain synchronization and regularly communicate at a specific interval. Synchronous event signaling allows the transmitter and receivers in each LP 102a, 102b to use limited (or minimal) power as each LP 102a, 102b is only powered for a small fraction of the time in connection with transmission and reception. For example, LP 102a, 102b may transmit/receive (Tx/Rx) communication messages in time slots having duration of 10-20 μs, where the Tx/Rx time slots occur periodically (e.g., every 10-20 ms). Such synchronous event signaling may be used, e.g., when the LPs 102a and 102b collectively provide DDD operation, but is not limited thereto.

LPs 102a and 102b may lose synchronization, even in a synchronous event signaling scheme. As explained herein, features may be included in LPs 102a and 102b to maintain device synchronization, and when synchronization is lost, LPs 102a and 102b undergo operations to recover synchronization. Also, synchronous event messages/signaling may introduce a delay between transmissions which causes a reaction lag at the receiving LP 102a, 102b. Accordingly, features may be implemented to account for the reaction lag.

During asynchronous event signaling, LPs 102a and 102b do not maintain communication synchronization. During asynchronous event signaling, one or more of receivers 120 and 122 of LPs 102a and 102b may be “always on” (always awake) to search for incoming transmissions. However, maintaining LP receivers 120, 122 in an “always on” (always awake) state presents challenges as the received signal level often is low due to high channel attenuation caused by the patient's anatomy. Further, maintaining the receivers awake will deplete the battery 114 more quickly than may be desirable.

In accordance with certain embodiments, the first receiver 120 may maintain the first channel active (awake) at all times (including when the second channel is inactive (asleep)) in order to listen for messages from a remote LP. The second receiver 122 may be assigned a second activation protocol that is a triggered protocol, in which the second receiver 122 becomes active (awake) in response to detection of trigger events over the first receive channel (e.g., when the incoming signal corresponds to the LP wakeup notice, activating the second channel at the local LP). The terms active, turned on, awake and enabled are used interchangeably herein.

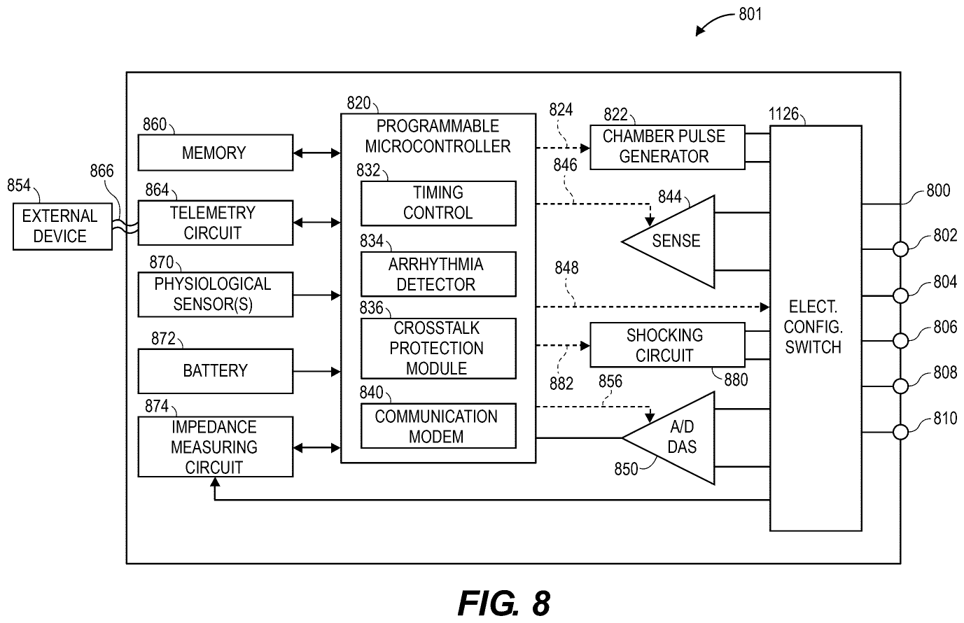

Still referring to FIG. 2, each LP 102a, 102b is shown as including a controller 112 and a pulse generator 116. The controller 112 can include, e.g., a microprocessor (or equivalent control circuitry), RAM and/or ROM memory, logic and timing circuitry, state machine circuitry, and I/O circuitry, but is not limited thereto. The controller 112 can further include, e.g., timing control circuitry to control the timing of the stimulation pulses (e.g., pacing rate, atrio-ventricular (AV) delay, atrial interconduction (AA) delay, or ventricular interconduction (VV) delay, etc.). Such timing control circuitry may also be used for the timing of refractory periods, blanking intervals, noise detection windows, evoked response windows, alert intervals, marker channel timing, crosstalk protection duration, and so on. The controller 112 can further include other dedicated circuitry and/or firmware/software components that assist in monitoring various conditions of the patient's heart and managing pacing therapies. For example, the controller 112 may include an arrhythmia detector, which can be similar to the arrhythmia detector 834 discussed below with reference to FIG. 8.

The controller 112 and the pulse generator 116 may be configured to transmit event messages, via the electrodes 108, in a manner that does not inadvertently capture the heart in the chamber where LP 102a, 102b is located, such as when the associated chamber is not in a refractory state. In addition, an LP 102a, 102b that receives an event message may enter an “event refractory” state (or event blanking state) following receipt of the event message. The event refractory/blanking state may be set to extend for a determined period of time after receipt of an event message in order to avoid the receiving LP 102a, 102b from inadvertently sensing another signal as an event message that might otherwise cause retriggering. For example, the receiving LP 102a, 102b may detect a measurement pulse from another LP 102a, 102b or the programmer 109.

Since the conductive communication receivers 120, 122 (or alternatively, just a single conductive communication receiver) and the pulse generator 116 are used to perform conductive communication, these components can be considered parts of a conductive communication transceiver 124. The conductive communication transceiver 124 can alternatively include alternative and/or additional components that enable an LP, to transmit and receive conductive communication messages with another LP and/or with another type of IMD, such as ICD 106 and/or an external device, such as the programmer 109. For an example, while the same pulse generator 116 can be used to produce pacing pulses (for use in pacing a patient's heart) and conductive communication pulses (for use in communicating with another LP, another type of IMD, and/or an external device), it would also be possible that the conductive communication transceiver 124 can include its own dedicated pulse generator. For another example, as noted above, it would also be possible for the conductive communication transceiver 124 to include only a single receiver, rather than both receivers 120, 122. Other variations are also possible and within the scope of the embodiments described herein.

Still referring to FIG. 2, the LP 102 is also shown as including an antenna 118 that is coupled to a radio frequency (RF) communication transceiver 134, which is configured to transmit and receive RF communication messages using an RF communication protocol, such as a Bluetooth protocol, WiFi protocol, Bluetooth low energy (BLE) protocol, Medical Device Radiocommunications Service (MedRadio) protocol, and/or the like. In certain embodiments, the antenna 118 can be integrated into a fixation mechanism (e.g., 205) of the LP, in which case the antenna can be referred to as a fixation antenna. An example implementation of a fixation antenna is disclosed in and described with reference to FIGS. 3-5 of U.S. Pat. No. 10,583,300, titled “Leadless implantable medical device with fixation antenna member,” which is incorporated by reference as if set forth fully herein. In other embodiments, the antenna 118 is separate and distinct from the fixation mechanism of the LP 102. The specific type, location and formfactor of the antenna may depend on the specific type and formfactor of the IMD.

The RF communication transceiver 134 consumes more battery power than the conductive communication transceiver 124. More generally, it is more power efficient from an LP 102 (or other type of IMD) to use conductive communication than to use RF communication to communicate with another LP 102 (or other type of IMD). Accordingly, in accordance with certain embodiments of the present technology, the LP 102 (or other type of IMD) is configured to primarily transmit and receive messages using conductive communication, and RF communication is used as a backup or auxiliary type of communication that can be used when conductive communication is deactivated (aka turned off) or is unsuccessful or otherwise deficient, as will be described in additional detail below.

It is possible that the LPs 102a, 102b are only capable of utilizing conductive communication for communicating with one another and/or other devices (such as a programmer 109 and/or ICD 106), in which case the antenna 118 and the RF communication transceiver 134 may be eliminated. It may also be the case that the LPs 102a, 102b are only configured to communicate using RF communication, and to not utilize conductive communication, in which case certain circuitry may be eliminated, such as the receivers 120, 122. Alternatively, or additionally, it is possible that the LPs 102a, 102b utilize another type of communication, such as inductive communication, in which case the LPs 102a, 102b can each include a respective inductive communication coil.

In accordance with certain embodiments herein, the programmer 109 may communicate over a programmer-to-LP channel, with LP 102a, 102b utilizing the same communication scheme. The external programmer 109 may listen to the event message transmitted between LP 102a, 102b and synchronize programmer to implant communication such that programmer 109 does not transmit communication messages until after an i2i messaging sequence is completed.

In some embodiments, each individual LP 102 can comprise a hermetic housing 110 configured for placement on or attachment to the inside or outside of a cardiac chamber and at least two leadless electrodes 108 proximal to the housing 110 and configured for bidirectional communication with at least one other device (e.g., an NV-ICD 106) within or outside the body.

FIG. 2 depicts a single LP 102 (e.g., the LP 102a or 102b) and shows the LP's functional elements substantially enclosed in a hermetic housing 110. The LP 102 has at least two electrodes 108 located within, on, or near the housing 110, for delivering pacing pulses to and sensing electrical activity from the muscle of the cardiac chamber, and for bidirectional communication with at least one other device within or outside the body. Hermetic feedthroughs 130, 131 conduct electrode signals through the housing 110. The housing 110 contains a primary battery 114 to supply power for pacing, sensing, and communication. The housing 110 also contains circuits 132 for sensing cardiac activity from the electrodes 108, receivers 120, 122 for receiving information from at least one other device via the electrodes 108, and the pulse generator 116 for generating pacing pulses for delivery via the electrodes 108 and also for transmitting information to at least one other device via the electrodes 108. The housing 110 can optionally contain circuits for monitoring device health, for example a battery current monitor 136 and a battery voltage monitor 138, and can contain circuits for controlling operations in a predetermined manner.

The electrodes 108 can be configured to communicate bidirectionally among the multiple LPs and/or the implanted ICD 106 to coordinate pacing pulse delivery and optionally other therapeutic or diagnostic features using messages that identify an event at an individual pacemaker originating the message and a pacemaker receiving the message react as directed by the message depending on the origin of the message. An LP 102a, 102b that receives the event message reacts as directed by the event message depending on the message origin or location. In some embodiments or conditions, the two or more leadless electrodes 108 can be configured to communicate bidirectionally among the one or more LPs 102 and/or the ICD 106 and transmit data including designated codes for events detected or created by an individual pacemaker. Individual pacemakers can be configured to issue a unique code corresponding to an event type and a location of the sending pacemaker.

In some embodiments, an individual LP 102a, 102b can be configured to deliver a pacing pulse with an event message encoded therein, with a code assigned according to pacemaker location and configured to transmit a message to one or more other LPs via the event message coded pacing pulse. The LP or pacemakers receiving the message are adapted to respond to the message in a predetermined manner depending on type and location of the event.

Moreover, information communicated on the incoming channel can also include an event message from another LP signifying that the other LP has sensed a heartbeat or has delivered a pacing pulse, and identifies the location of the other pacemaker. For example, LP 102b may receive and relay an event message from LP 102a to the programmer. Similarly, information communicated on the outgoing channel can also include a message to another LP or LPs, or to the ICD, that the sending LP has sensed a heartbeat or has delivered a pacing pulse at the location of the sending pacemaker.

Referring again to FIGS. 1 and 2, the system 100 may comprise an ICD 106 in addition to one or more LPs 102a, 102b configured for implantation in electrical contact with a cardiac chamber and for performing cardiac rhythm management functions in combination with the implantable ICD 106. The implantable ICD 106 and the one or more LPs 102a, 102b configured for leadless intercommunication by information conduction through body tissue and/or wireless transmission between transmitters and receivers in accordance with the embodiments discussed herein.

In a further embodiment, the system 100 comprises at least one LP 102a, 102b configured for implantation in electrical contact with a cardiac chamber and configured to perform cardiac pacing functions in combination with the co-implanted ICD 106. Each LP 102 comprise at least two leadless electrodes 108 configured for delivering cardiac pacing pulses, sensing evoked and/or natural cardiac electrical signals, and transmitting information to the co-implanted ICD 106.

As shown in the illustrative embodiments, an LP 102a, 102b can comprise two or more leadless electrodes 108 configured for delivering cardiac pacing pulses, sensing evoked and/or natural cardiac electrical signals, and bidirectionally communicating with the co-implanted ICD 106.

Each LP 102a, 102b can be configured for operation in a respective particular location and to have a respective particular functionality at manufacture and/or by programming by an external programmer. Bidirectional communication among the multiple LPs can be arranged to communicate notification of a sensed heartbeat or delivered pacing pulse event and encoding type and location of the event to another implanted pacemaker or pacemakers. The LP 102a, 102b receiving the communication decode the information and respond depending on location of the receiving pacemaker and predetermined system functionality.

In some embodiments, the LPs 102a and 102b are configured to be implantable in any chamber of the heart, namely either atrium (RA, LA) or either ventricle (RV, LV). Furthermore, for dual-chamber configurations, multiple LPs may be co-implanted (e.g., one in the RA and one in the RV, one in the RV and one in the coronary sinus proximate the LV). Certain pacemaker parameters and functions depend on (or assume) knowledge of the chamber in which the LP is implanted (and thus with which the LP is interacting; e.g., pacing and/or sensing). Some non-limiting examples include an evoked response algorithm, use of atrial fibrillation (AF) suppression in a local chamber, blanking & refractory periods, etc. Accordingly, each LP should know an identity of the chamber in (or on) which the LP is implanted, and processes may be implemented to automatically identify a local chamber associated with each LP.

Processes for chamber identification may also be applied to subcutaneous pacemakers, ICDs, with leads and the like. A device with one or more implanted leads, identification and/or confirmation of the chamber into which the lead was implanted could be useful in several pertinent scenarios. For example, for a dual chamber rate responsive pacing device or a cardiac resynchronization therapy (CRT) device, automatic identification and confirmation could militate against the possibility of the clinician inadvertently placing the V lead into the A port of the implantable medical device, and vice-versa. As another example, for a single chamber rate responsive pacing device, automatic identification of implanted chamber could enable the device and/or programmer to select and present the proper subset of pacing modes (e.g., AAI or VVI), and for the implantable device to utilize the proper set of settings and algorithms (e.g., V-AutoCapture vs ACap-Confirm, sensing sensitivities, etc.).

In an embodiment, the primary battery 114 of FIG. 2 has positive terminal 140 and negative terminal 142. Current from the positive terminal 140 of primary battery 114 flows through an optional shunt 144 to an optional regulator circuit 146 to create a positive voltage supply 148 suitable for powering the remaining circuitry of the LP 102. The shunt 144 enables the optional battery current monitor 136 to provide the controller 112 with an indication of battery current drain and indirectly of device health. The illustrative power supply can be a primary battery 114.

Still referring to FIG. 2, the LP is shown as including an optional temperature sensor 152. The temperature sensor can be any one of various types of well-known temperature sensors, or can be a future developed temperature sensor. For one example, the temperature sensor 152 can be a thermistor, a thermocouple, a resistance thermometer, or a silicon bandgap temperature sensor, but is not limited thereto. Regardless of how the temperature sensor 152 is implemented, it is preferably that the temperature sensed by the sensor is provided to the controller 112 as a digital signal indicative of the blood temperature of the patient within which the LP is implanted. The temperature sensor 152 can be hermetically sealed within the housing 110, but that need not be the case. The temperature sensor 152 can be used in various manners. For example, the temperature sensor 152 can be used to detect an activity level of the patient to adjust a pacing rate, i.e., for use in rate responsive pacing. When a person starts to exercise their core body temperature initially dips, and then after exercising for a prolonged period of time the person's core body temperature will eventually rise. Thereafter, when the person stops exercising their core body temperature will return to its baseline. Accordingly, the controller 112 can be configured to detect an activity level of a patient based on core blood temperature measurements obtained using the temperature sensor 152.

Referring to FIG. 2, the LP is also shown as including an optional accelerometer 154 which can be hermetically contained within the housing 110. The accelerometer 154 can be any one of various types of well-known accelerometers or can be a future developed accelerometer. For one example, the accelerometer 154 can be or include, e.g., a MEMS (micro-electromechanical system) multi-axis accelerometer of the type exploiting capacitive or optical cantilever beam techniques, or a piezoelectric accelerometer that employs the piezoelectric effect of certain materials to measure dynamic changes in mechanical variables. For example, the accelerometer 154 can be used to detect an activity level of the patient to adjust a pacing rate, i.e., for use in rate responsive pacing. It would also be possible to use outputs of both the accelerometer 154 and the temperature sensor 152 to monitor the activity level of a patient. Alternatively, or additionally, a patient's activity level can be monitored based on their heart rate, as detected from an electrogram (EGM) sensed using the electrodes 108, and/or sensed using a plethysmography signal obtained using a plethysmography sensor (not shown) or a heart sound sensor (not shown), but not limited thereto. One or more signals produced and output by the accelerometer 154 may be analyzed with respect to frequency content, energy, duration, amplitude and/or other characteristics. Such signals may or may not be amplified and/or filtered prior to being analyzed. For example, filtering may be performed using lowpass, highpass and/or bandpass filters. The signals output by the accelerometer 154 can be analog signals, which can be analyzed in the analog domain, or can be converted to digital signals (by an analog-to-digital converter) and analyzed in the digital domain. Alternatively, the signals output by the accelerometer 154 can already be in the digital domain. The one or more signals output by the accelerometer 154 can be analyzed by the controller 112 and/or other circuitry. In certain embodiments, the accelerometer 154 is packaged along with an integrated circuit (IC) that is designed to analyze the signal(s) it generates. In such embodiments, one or more outputs of the packaged sensor/IC can be an indication of acceleration along one or more axes. In other embodiments, the accelerometer 154 can be packaged along with an IC that performs signal conditioning (e.g., amplification and/or filtering), performs analog-to-digital conversions, and stores digital data (indicative of the sensor output) in memory (e.g., RAM, which may or may not be within the same package). In such embodiments, the controller 112 or other circuitry can read the digital data from the memory and analyze the digital data. Other variations are also possible, and within the scope of embodiments of the present technology. In accordance with certain embodiments of the present technology, described in additional detail below, a sensor signal produced by the accelerometer 154 of an LP implanted in or on a cardiac chamber can be used to detect mechanical cardiac activity associated with another cardiac chamber.

In various embodiments, LP 102a, 102b can manage power consumption to draw limited power from the battery, thereby reducing device volume. Each circuit in the LP 102 can be designed to avoid large peak currents. For example, cardiac pacing can be achieved by discharging a tank capacitor (not shown) across the pacing electrodes. Recharging of the tank capacitor is typically controlled by a charge pump circuit. In a particular embodiment, the charge pump circuit is throttled to recharge the tank capacitor at constant power from the battery.

In some embodiments, the controller 112 in an LP 102 can access signals on the electrodes 108 and can examine output pulse duration from another LP 102 for usage as a signature for determining triggering information validity and, for a signature arriving within predetermined limits, activating delivery of a pacing pulse following a predetermined delay of zero or more milliseconds. The predetermined delay can be preset at manufacture, programmed via an external programmer, or determined by adaptive monitoring to facilitate recognition of the triggering signal and discriminating the triggering signal from noise. In some embodiments or in some conditions, the controller 112 can examine output pulse waveform from another LP for usage as a signature for determining triggering information validity and, for a signature arriving within predetermined limits, activating delivery of a pacing pulse following a predetermined delay of zero or more milliseconds.

FIG. 3 shows an example form factor of the LPs 102a, 102b. Each LP can include a hermetic housing 202 (e.g., 110 in FIG. 2) with electrodes 108a and 108b disposed thereon. As shown, electrode 108a can be separated from but surrounded partially by a fixation mechanism 205, and the electrode 108b can be disposed on the housing 202. The fixation mechanism 205 can be a fixation helix, a plurality of hooks, barbs, or other attaching features configured to attach the LP to tissue, such as heart tissue. As noted above, an antenna (e.g., 118) can be at least partially implanted by or as part of the fixation mechanism. The electrodes 108a and 108b are examples of the electrodes 108 shown in and discussed above with reference to FIG. 2.

The housing can also include an electronics compartment 210 within the housing that contains the electronic components necessary for operation of the LP, including, e.g., a pulse generator, transceiver, a battery, and a processor for operation. The hermetic housing 202 can be adapted to be implanted on or in a human heart, and can be cylindrically shaped, rectangular, spherical, or any other appropriate shapes, for example.

The housing can comprise a conductive, biocompatible, inert, and anodically safe material such as titanium, 316L stainless steel, or other similar materials. The housing can further comprise an insulator disposed on the conductive material to separate electrodes 108a and 108b. The insulator can be an insulative coating on a portion of the housing between the electrodes, and can comprise materials such as silicone, polyurethane, parylene, or another biocompatible electrical insulator commonly used for implantable medical devices. In the embodiment of FIG. 3, a single insulator 208 is disposed along the portion of the housing between electrodes 108a and 108b. In some embodiments, the housing itself can comprise an insulator instead of a conductor, such as an alumina ceramic or other similar materials, and the electrodes can be disposed upon the housing.

As shown in FIG. 3, the LP can further include a header assembly 212 to isolate 108a and 108b. The header assembly 212 can be made from PEEK, tecothane or another biocompatible plastic, and can contain a ceramic to metal feedthrough, a glass to metal feedthrough, or other appropriate feedthrough insulator as known in the art.

The electrodes 108a and 108b can comprise pace/sense electrodes, or return electrodes. A low-polarization coating can be applied to the electrodes, such as sintered platinum, platinum-iridium, iridium, iridium-oxide, titanium-nitride, carbon, or other materials commonly used to reduce polarization effects, for example. In FIG. 3, electrode 108a can be a pace/sense electrode and electrode 108b can be a return electrode. The electrode 108b can be a portion of the conductive housing 202 that does not include an insulator 208.

Several techniques and structures can be used for attaching the housing 202 to the interior or exterior wall of the heart. A helical fixation mechanism 205, can enable insertion of the device endocardially or epicardially through a guiding catheter. A torqueable catheter can be used to rotate the housing and force the fixation device into heart tissue, thus affixing the fixation device (and also the electrode 108a in FIG. 3) into contact with stimulable tissue. Electrode 108b can serve as an indifferent electrode for sensing and pacing. The fixation mechanism may be coated partially or in full for electrical insulation, and a steroid-eluting matrix may be included on or near the device to minimize fibrotic reaction, as is known in conventional pacing electrode-leads.

### Implant-to-Implant Event Messaging

LPs 102a and 102b can utilize implant-to-implant (i2i) communication through event messages to coordinate operation with one another in various manners. The terms i2i event messages and i2i event markers are used interchangeably herein to refer to event related messages and LP/LP operation related messages transmitted from an implanted device and directed to another implanted device (although external devices, e.g., a programmer, may also receive i2i event messages). In certain embodiments, LP 102a and LP 102b operate as two independent leadless pacers maintaining beat-to-beat dual-chamber functionality via a “Master/Slave” operational configuration. For descriptive purposes, the ventricular LP 102b shall often be referred to as “vLP” and the atrial LP 102a shall often be referred to as “aLP,” as was noted above. The LP 102 that is designated as the master device (e.g., vLP) may implement all or most dual-chamber diagnostic and therapy determination algorithms. For purposes of the following illustration, it is assumed that the vLP is a “master” device, while the aLP is a “slave” device. Alternatively, the aLP may be designated as the master device, while the vLP may be designated as the slave device. The master device orchestrates most or all decision-making and timing determinations (including, for example, rate-response changes).

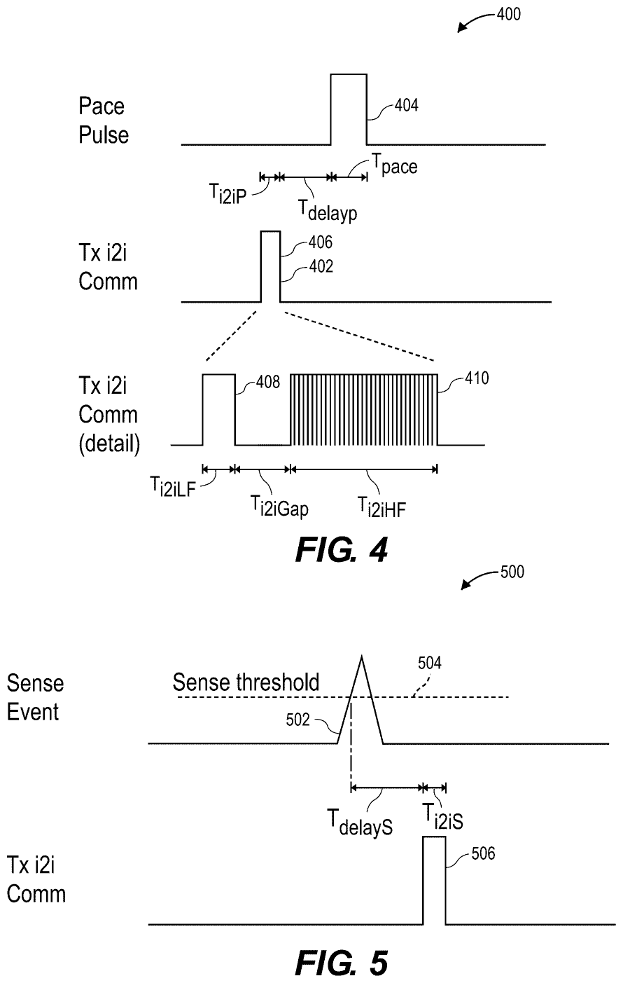

FIG. 4 is a timing diagram 400 demonstrating one example of an i2i conductive communication for a paced event. The i2i conductive communication may be transmitted, for example, from LP 102a to LP 102b. As shown in FIG. 4, in this embodiment, an i2i transmission 402 is sent prior to delivery of a pace pulse 404 by the transmitting LP (e.g., LP 102a). This enables the receiving LP (e.g., LP 102b) to prepare for the remote delivery of the pace pulse. The i2i transmission 402 includes an envelope 406 that may include one or more individual pulses. For example, in this embodiment, envelope 406 includes a low frequency pulse 408 followed by a high frequency pulse train 410. Low frequency pulse 408 lasts for a period T_{i2iLF}, and high frequency pulse train 410 lasts for a period T_{i2iHF}. The end of the low frequency pulse 408 and the beginning of the high frequency pulse train 410 are separated by a gap period, T_{i2iGap}. In alternative embodiments, rather than transmitting the envelope 406 prior to the pacing pulse 404, the envelope 406 can be transmitted during a refractory period that follows the delivery of the pacing pulse.

As shown in FIG. 4, the i2i transmission 402 lasts for a period Ti2iP, and pace pulse 404 lasts for a period Tpace. The end of i2i transmission 402 and the beginning of pace pulse 404 are separated by a delay period, TdelayP. The delay period may be, for example, between approximately 0.0 and 10.0 milliseconds (ms), particularly between approximately 0.1 ms and 2.0 ms, and more particularly approximately 1.0 ms. The term approximately, as used herein, means+/−10% of a specified value.

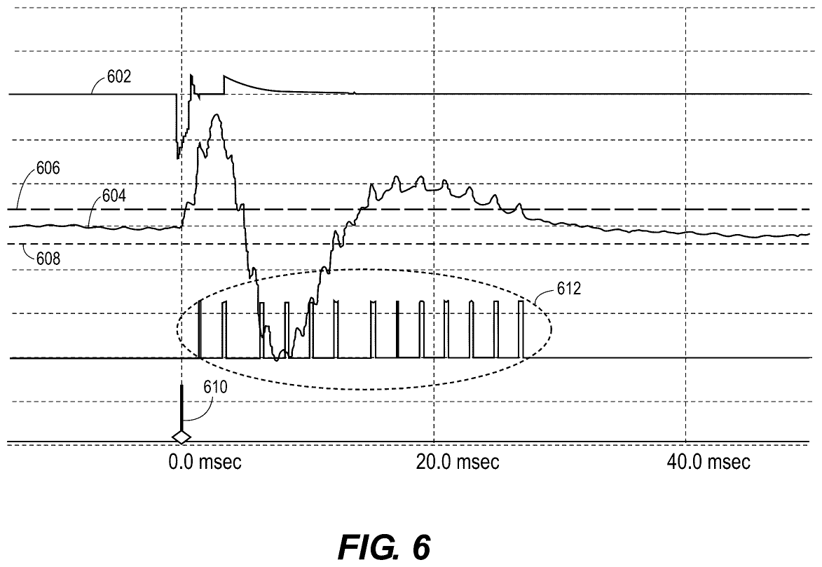

FIG. 5 is a timing diagram 500 demonstrating one example of an i2i communication for a sensed event. The i2i communication may be transmitted, for example, from LP 102a to LP 102b. As shown in FIG. 5, in this embodiment, the transmitting LP, e.g., LP 102a detects the sensed event when a sensed intrinsic activation 502 crosses a sense threshold 504. A predetermined delay period, T_{delayS}, after the detection, the transmitting LP transmits an i2i transmission 506 that lasts a predetermined period T_{i2iS}. The delay period may be, for example, between approximately 0.0 and 10.0 milliseconds (ms), particularly between approximately 0.1 ms and 2.0 ms, and more particularly approximately 1.0 ms.

As with i2i transmission 402, i2i transmission 506 may include an envelope that may include one or more individual pulses. For example, similar to envelope 406, the envelope of i2i transmission 506 may include a low frequency pulse followed by a high frequency pulse train. In certain embodiments, the i2i transmission 506 is transmitted during a refractory period that follows the sensed event.

Optionally, wherein the first LP is located in an atrium and the second LP is located in a ventricle, the first LP produces an AS/AP event marker to indicate that an atrial sensed (AS) event or atrial paced (AP) event has occurred or will occur in the immediate future. For example, the AS and AP event markers may be transmitted following the corresponding AS or AP event. Alternatively, the first LP may transmit the AP event marker slightly prior to delivering an atrial pacing pulse. Alternatively, wherein the first LP is located in an atrium and the second LP is located in a ventricle, the second LP initiates an atrioventricular (AV) interval after receiving an AS or AP event marker from the first LP; and initiates a post atrial ventricular blanking (PAVB) interval after receiving an AP event marker from the first LP.

Optionally, the first and second LPs may operate in a “pure” master/slave relation, where the master LP delivers “command” markers in addition to or in place of “event” markers. A command marker directs the slave LP to perform an action such as to deliver a pacing pulse and the like. For example, when a slave LP is located in an atrium and a master LP is located in a ventricle, in a pure master/slave relation, the slave LP delivers an immediate pacing pulse to the atrium when receiving an AP command marker from the master LP.

In accordance with some embodiments, communication and synchronization between the aLP and vLP is implemented via conducted communication of markers/commands in the event messages (per i2i communication protocol). As explained above, conducted communication represents event messages transmitted from the sensing/pacing electrodes at frequencies outside the RF (e.g., Wi-Fi or BLE) frequency range. Alternatively, the event messages may be conveyed over communication channels operating in the RF frequency range. The figures and corresponding description below illustrate non-limiting examples of markers that may be transmitted in event messages. The figures and corresponding description below also include the description of the markers and examples of results that occur in the LP that receives the event message. Table 1 represents example event markers sent from the aLP to the vLP, while Table 2 represents example event markers sent from the vLP to the aLP. In the master/slave configuration, AS event markers are sent from the aLP each time that an atrial event is sensed outside of the post ventricular atrial blanking (PVAB) interval or some other alternatively-defined atrial blanking period. The AP event markers are sent from the aLP each time that the aLP delivers a pacing pulse in the atrium. The aLP may restrict transmission of AS markers, whereby the aLP transmits AS event markers when atrial events are sensed both outside of the PVAB interval and outside the post ventricular atrial refractory period (PVARP) or some other alternatively-defined atrial refractory period. Alternatively, the aLP may not restrict transmission of AS event markers based on the PVARP, but instead transmit the AS event marker every time an atrial event is sensed.

TABLE 1
“A2V” Markers/Commands (i.e., from aLP to vLP)

| Marker | Description | Result in vLP |
|---|---|---|
| AS | Notification of a sensed event in | Initiate AV interval (if not |
| | atrium (if not in PVAB or PVARP) | in PVAB or PVARP) |
| AP | Notification of a paced event in | Initiate PAVB |
| | atrium | Initiate AV interval (if not |
| | | in PVARP) |

As shown in Table 1, when an aLP transmits an event message that includes an AS event marker (indicating that the aLP sensed an intrinsic atrial event), the vLP initiates an AV interval timer. If the aLP transmits an AS event marker for all sensed events, then the vLP would preferably first determine that a PVAB or PVARP interval is not active before initiating an AV interval timer. If however the aLP transmits an AS event marker only when an intrinsic signal is sensed outside of a PVAB or PVARP interval, then the vLP could initiate the AV interval timer upon receiving an AS event marker without first checking the PVAB or PVARP status. When the aLP transmits an AP event marker (indicating that the aLP delivered or is about to deliver a pace pulse to the atrium), the vLP initiates a PVAB timer and an AV interval time, provided that a PVARP interval is not active. The vLP may also blank its sense amplifiers to prevent possible crosstalk sensing of the remote pace pulse delivered by the aLP.

TABLE 2
“V2A” Markers/Commands (i.e., from vLP to aLP)

| Marker | Description | Result in aLP |
|---|---|---|
| VS | Notification of a sensed event in | Initiate PVARP |
| | ventricle | |
| VP | Notification of a paced event in | Initiate PVAB |
| | ventricle | Initiate PVARP |
| AP | Command to deliver immediate pace | Deliver immediate pace |
| | pulse in atrium | pulse to atrium |

As shown in Table 2, when the vLP senses a ventricular event, the vLP transmits an event message including a VS event marker, in response to which the aLP may initiate a PVARP interval timer. When the vLP delivers or is about to deliver a pace pulse in the ventricle, the vLP transmits VP event marker. When the aLP receives the VP event marker, the aLP initiates the PVAB interval timer and also the PVARP interval timer. The aLP may also blank its sense amplifiers to prevent possible crosstalk sensing of the remote pace pulse delivered by the vLP. The vLP may also transmit an event message containing an AP command marker to command the aLP to deliver an immediate pacing pulse in the atrium upon receipt of the command without delay.

The foregoing event markers are examples of a subset of markers that may be used to enable the aLP and vLP to maintain full dual chamber functionality. In one embodiment, the vLP may perform all dual-chamber algorithms, while the aLP may perform atrial-based hardware-related functions, such as PVAB, implemented locally within the aLP. In this embodiment, the aLP is effectively treated as a remote ‘wireless’ atrial pace/sense electrode. In another embodiment, the vLP may perform most but not all dual-chamber algorithms, while the aLP may perform a subset of diagnostic and therapeutic algorithms. In an alternative embodiment, vLP and aLP may equally perform diagnostic and therapeutic algorithms. In certain embodiments, decision responsibilities may be partitioned separately to one of the aLP or vLP. In other embodiments, decision responsibilities may involve joint inputs and responsibilities.

Messages that are transmitted between LPs (e.g., the aLP and the vLP) can be referred to herein generally as i2i messages, since they are implant-to-implant messages. As noted above, such messages can include event markers that enable one LP to inform the other LP of a paced event or a sensed event. For example, in certain embodiments, whenever the aLP 102a senses an atrial event or paces the right atrium, the aLP will transmit an i2i message to the vLP 102b to inform the vLP of the sensed or paced event in the atrium. In response to receiving such an i2i message, the vLP 102b may start one or more timers that enable the vLP to sense or pace in the right ventricle. Similarly, the vLP may transmit an i2i message to the aLP 102a whenever the vLP senses a ventricular event or paces the right ventricle.

The i2i messages that are sent between LPs may be relatively short messages that simply allow a first LP to inform a second LP of an event that was sensed by the first LP or caused (paced) by the first LP, and vice versa. Such i2i messages can be referred to herein as event marker i2i messages, or more succinctly as event i2i messages. The i2i messages that are sent between LPs, in certain instances, can be extended i2i messages that include (in addition to an event marker) an extension. In certain embodiments, an extended i2i message includes an event marker (e.g., 9 bits), followed by an extension indicator (e.g., 2 bits), followed by an extended message payload portion (e.g., 17 bits), followed by a cyclic redundancy check (CRC) code (e.g., 6 bits) or some other type of error detection and correction code. Other variations are also possible.

### Crosstalk Protection

As explained above, to mitigate against (and preferably prevent) crosstalk adversely affecting a dual chamber LP system (or more generally, a multi-chamber LP system), each LP of the system can independently monitor for possible crosstalk that may be caused by another LP delivering a pacing pulse. Then, in response to detecting possible crosstalk, the LP that detects the possible crosstalk can blank for a period of time its sense circuit, which sense circuit the LP uses to monitor for intrinsic events of the chamber in (or on) which the LP is implanted. Alternatively, or additionally, in response to detecting possible crosstalk, the LP that detects the crosstalk can ignore for a period of time any possible intrinsic events that its sense circuit detects, under the assumption that such detections of the intrinsic event(s) was/were actually due to the crosstalk. More generally, the LP that detects the possible crosstalk can initiate crosstalk protection in response to detecting the possible crosstalk.

As noted above, determining an appropriate crosstalk protection duration for use in a dual chamber LP system (and more generally, a multi-chamber LP system) is not a trivial endeavor, as there are various factors of an LP system that may affect the appropriate crosstalk protection duration. If a crosstalk protection duration is set too short, that may lead to a residual detection of crosstalk. Conversely, if a crosstalk protection duration is set too long, that may lead to legitimate local intrinsic events being missed (i.e., failing to be detected). Certain embodiments of the present technology, which will now be described in additional detail below, relate to systems, subsystems, and methods that specify and utilize an appropriate crosstalk protection duration for an LP of a multi-chamber LP system.

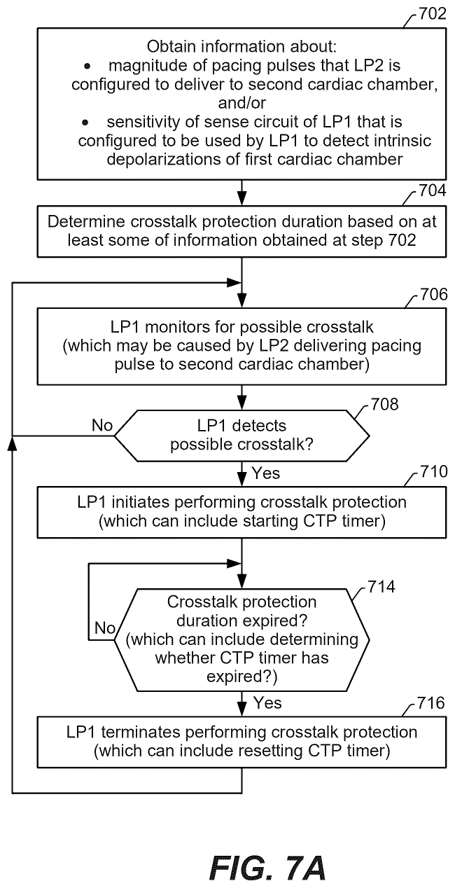

FIG. 6 is an example timing diagram that is used to illustrate how a pacing pulse delivered by a second LP (LP2) implanted in (or on) a remote cardia chamber (e.g., the right atrium) may cause crosstalk that may be falsely detected as an intrinsic depolarization of a local cardiac chamber by a first LP (LP1) implanted in (or on) the local cardiac chamber (e.g., the right ventricle). The waveform 602 is a signal sensed between a pair of electrodes (e.g., 108a and 108b) of the LP1, before any filtering is performed. The waveform 604 is the sense signal 602 after it has been filtered by the sense circuit (e.g., 132) of the LP1 that is used by the LP1 to monitor for intrinsic depolarizations of the local cardiac chamber in (or on) which the LP1 is implanted. It is noted that the vertical (aka y-axis) scalings of the signals 602 and 604 are not equivalent, i.e., the signal 604 is magnified in the y-axis relative to the signal 602. The horizontal dashed lines 606 and 608 are respectively positive and negative intrinsic depolarization sensing thresholds (e.g., at +1.0 mv and −1.0 mv, or at +0.5 mV and −0.5 mV, but not limited thereto) that are used by the sense circuit (e.g., 132) of the LP1 to detect intrinsic depolarizations of the local cardiac chamber (e.g., the right ventricle) when a magnitude of the filtered signal 604 is beyond the intrinsic depolarization sensing thresholds (represented by the horizontal dashed lines 606 and 608). The pulse 610 represents when possible crosstalk is detected by the LP1 (e.g., by the LF receiver 120 of the LP1). In other words, the pulse 610 represents the timing of when possible crosstalk is detected by the LP1. As will be described in additional detail below, it is possible that the possible crosstalk detected by the LP1 is not crosstalk, but rather, is actually a portion (e.g., a wakeup pulse of) a valid message received from another device. The pulses within the dotted oval 612 are interrupt pulses that are generated no more frequently than once per clock cycle (of a clock of the LP1) when the filtered signal 604 is beyond the intrinsic depolarization sensing thresholds (represented by the horizontal dashed lines 606 and 608). Notice that no interrupt pulses occur when a magnitude of the filtered signal 604 is not beyond the intrinsic depolarization sensing thresholds. While both positive and negative intrinsic depolarization sensing thresholds are shown in FIG. 6, it is possible to rectify the filtered signal 604 so that it is always a positive signal, in which case there is only a need to use the positive intrinsic depolarization sensing threshold 606.

As can be appreciated from FIG. 6, if the crosstalk protection duration is set to about 30 msec, then all the interrupts (within the dotted oval 612) would be ignored (or not produced, depending upon the implementation) following the possible crosstalk being detected (as represented by the pulse 610). However, if the crosstalk protection duration was set too short, e.g., to 20 msec, then the LP1 would still mistakenly detect a possible intrinsic depolarization of the local chamber in (or on) which the LP1 is implanted, due to ringing of the filtered signal 604 that still has a magnitude beyond the intrinsic depolarization sensing thresholds (represented by the horizontal dashed lines 606 and 608) after the end of that too short crosstalk protection duration. On the other hand, if the crosstalk protection duration was set too long, e.g., to 50 msec, there is an increased probability that the LP1 will fail to detect an actual intrinsic depolarization of the local chamber in (or on) which the LP1 is implanted.

In accordance with certain embodiments of the present technology, a crosstalk protection duration that is for use by an LP is determined based on certain information about the multi-chamber LP system of which the LP is included. For the purpose of the following discussion, such a multi-chamber LP system is assumed to include a first leadless pacemaker (LP1) (e.g., 102a) that is configured to be implanted in or on a first cardiac chamber (e.g., the right atrium) of a patient's heart and to deliver pacing pulses to the first cardiac chamber, and a second leadless pacemaker (LP2) (e.g., 102b) that is configured to be implanted in or on a second cardiac chamber (e.g., the right ventricle) of the patient's heart and to deliver pacing pulses to the second cardiac chamber. In such a system, it is also assumed that each LP includes a respective sense circuit (e.g., 132) that is configured to be used by the LP to detect intrinsic depolarizations of the local cardiac chamber in (or on) which the LP is implanted. As explained below, each LP can also include a respective crosstalk detection circuit that is configured to be used by the LP to detect potential crosstalk that may be caused by another LP delivering one of the pacing pulses. Additionally, it is assumed that each LP includes a respective pulse generator (e.g., 116) that is configured to be used by the LP to produce pacing pulses that are delivered to the local cardiac chamber in (or on) which the LP is implanted. Such a pulse generator may include one or more pacing capacitor(s) and one or more return capacitor(s), that collectively have a capacitance (Cpace) and are configured to store and deliver energy used to produce the pacing pulses.

For the purpose of this discussion, the cardiac chamber within or on which a particular LP is implanted can be referred to as a “local chamber”, while another chamber (within or on which the particular LP is not implanted) can be referred to as a “remote chamber”. From the perspective of an LP implanted in or on a local cardiac chamber (e.g., the right atrium), simulations and tests have shown that a magnitude of a pacing pulse delivered to a remote cardiac chamber (e.g., the right ventricle), by another LP implanted in or on the remote cardiac chamber, is positively correlated with how long crosstalk (caused by the other LP delivering a pacing pulse to the remote chamber) is detectable to the LP implanted in or on a local cardiac chamber (e.g., the right atrium), wherein the local cardiac chamber is the cardiac chamber within (or on) which a particular LP is implanted, as was noted above. The magnitude of the pacing pulse can be quantified, e.g., in terms of its pacing pulse amplitude (PPA) and/or its pacing pulse width (P_{PW}), and preferably both. More specifically, a viable first-order approximation of the magnitude of a pacing pulse, and more specifically, the magnitude of the delivered pacing pulse charge (P_{PQ}), is P_{PA}×P_{PW}. An even more accurate approximation for the pacing pulse charge (P_{PQ}) also takes into account a capacitance (C_{pace}) of the one or more pacing capacitor(s) of the remote LP that store energy used to produce the pacing pulses delivered to the remote chamber, as well as the pace output impedance (R_{load}), using the following equation (in which equation (EQ1) the coefficient 1000 is used to scale the numerator to match the units of the denominator, and thus, is dependent on the specific implementation, and in certain implementations may be different or eliminated):

$${P}_{PQ} = {C}_{pace} \cdot {P}_{PA} \cdot ( 1 - exp [ \frac{- ( 1000 \cdot {P}_{PW} )}{( {R}_{load} \cdot {C}_{pace} )} ] ) \qquad ( EQ 1 )$$

Additionally, simulations and tests have also shown that a sensitivity of the sense circuit (e.g., 132) of the LP implanted in or on a local cardiac chamber (e.g., the right atrium) is negatively correlated with how long crosstalk (caused by the other LP delivering a pacing pulse to the remote chamber) is detectable by the LP implanted in or on the local cardiac chamber (e.g., the right atrium), assuming the sensitivity is specified by a sense detection threshold. If the sensitivity of the sense circuit (e.g., 132) is instead specified by a gain of the sense circuit, while the sense detection threshold remains constant, then the sensitivity (and more specially, gain) is instead positively correlated with how long crosstalk (caused by the other LP delivering a pacing pulse to the remote chamber) is detectable the LP implanted in or on the local cardiac chamber (e.g., the right atrium).

Further, bench testing in a saline bath was performed and the results were analyzed to create a set of mathematical crosstalk protection models or equations that captured the dependency of crosstalk protection duration on the pacing pulse magnitude from a remote LP (through the parameters pulse amplitude and pulse width) and the sense detection threshold of the sense circuit (e.g., 132) of the local LP, assuming a fixed distance and orientation between those LPs.

Based on the results of the bench testing, the following equation (aka model) was derived for specifying the crosstalk protection duration to be used by an LP (e.g., 102b) implanted in a right ventricle (RV), wherein another LP (e.g., 102a) is implanted in a right atrium (RA):

$$i 2 {iCTP}_{RV} = {K}_{m} \cdot ln ( \frac{{P}_{PQ \_ RA}}{{Sens}_{RV}} ) + {K}_{b} \qquad ( EQ 2 )$$

where,
i2iCTP_{RV} is the crosstalk protection duration to be used by the LP implanted in the RV,
Kₘ is a constant scaling factor determined for a patient population,
K_{b} is a constant offset factor determined for the patient population,
P_{PQ_RA} is the pacing pulse charge of each pacing pulse delivered by the LP implanted in the RA,
Sens_{RV} is the sense detection threshold of the sense circuit of the LP implanted in the RV, and
ln is the natural log function.

Additionally, based on the results of the bench testing, the following equation (aka model) was derived for specifying the crosstalk protection duration to be used by a LP (e.g., 102a) implanted in an RA, wherein another LP (e.g., 102b) is implanted in an RV:

$$i 2 {iCTP}_{RA} = [ {K}_{m} \cdot ln ( \frac{{P}_{PQ \_ RV}}{{Sens}_{RA}} ) + {K}_{b} ] + [ {K}_{a} \cdot sech ( {K}_{c} \cdot \{ ln ( \frac{{P}_{PQ \_ RV}}{{Sens}_{RA}} ) - {X}_{0} \} ) ] \qquad ( EQ 3 )$$

where,
i2iCTP_{RA} is the crosstalk protection duration to be used by the LP implanted in the RA,
Kₘ is the constant scaling factor determined for the patient population,
K_{b} is the constant offset factor determined for the patient population,
K_{c} is a further constant scaling factor determined for the patient population,
X₀ is a further constant offset factor determined for the patient population,
P_{PQ_RV} is the pacing pulse charge of each pacing pulse delivered by the LP implanted in the RV,
Sens_{RA} is the sense detection threshold of the sense circuit of the LP implanted in the RA,
ln is the natural log function, and
sech is the hyperbolic secant function.

For one particular analysis and implementation, values for Kₘ, K_{b}, Kₐ, K_{c}, and X₀ were defined as shown below:
Kₘ=9,
K_{b}=−3,
Kₐ=8,
K_{c}=2, and
X₀=1.9.

Embodiments of the present technology described herein are not limited to uses of the specified equations described above. However, such equations can be used in specific embodiments of the present technology. In other words, such equations are specific examples of a myriad of equations that may be used to implement various embodiments described herein.

In addition, in accordance with certain embodiments, a further scaling factor that is related to a distance and/or angle between the first and second LPs may be used to further enhance/refine the crosstalk protection duration. Determination of an appropriate scaling factor may be established by the LPs automatically via various means, or it may be manually configured by a user during programming. Such a scaling factor can, e.g., have a negative correlation between the distance between the LPs and the crosstalk protection duration. Additionally, or alternatively, the scaling factor can have a negative correlation between the relative angle between the LPs and the crosstalk protection duration. That is, the crosstalk protection duration would decrease as the relative angle between LP increases, wherein if the LPs are parallel to one another there is considered to be a zero angle between the LPs. Once the scaling factor is determined, it can be multiplied by the crosstalk protection duration determination that was determined based on a magnitude of the pacing pulses that the LP in the remote chamber delivers and/or based on the sense detection threshold of the LP in the local chamber, e.g., using one of the equations discussed above. In certain embodiments, the scaling factor (sf) can have a value between 0 and 1, i.e., 0<sf<1.

In accordance with certain embodiments, the scaling factor is selected by a user (clinician or physician) and programmed using a programmer type of external device. In other embodiments, a controller of an LP (or of an external device, e.g., programmer) can automate the determination of the scaling factor that is to be used by that LP in which the controller is included. For example, a crosstalk protection duration can be determined in one of the above described manners, assuming a predetermined distance between (e.g., 2 cm) and a predetermined angle (e.g., zero degrees) between the LP1 and the LP2. A functional relationship between the LP1 sensed amplitude and distance between the LP1 and LP2 (at zero degrees, or some other predetermined angle) is established in-vitro for an LP2 pace pulse having a known magnitude (e.g., a 6 V amplitude, and a 0.4 ms pulse width), and the relationship is determined by and/or provided to the controller of the LP1. Then, after the LP1 and the LP2 are implanted, the LP1 measures the in-vivo sensed amplitude of detected crosstalk caused by the LP2 delivering a pacing pulse having the known magnitude (e.g., a 6V pacing pulse amplitude, and a 0.4 ms pacing pulse width). The controller of the LP1 can then compare the in-vivo sensed amplitude to the in-vitro sensed amplitude (measured when there was a known distance, e.g., 2 cm, between the LP1 and LP2, and the angle between the LP1 and the LP2 was zero degrees, or some other known predetermined angle) to determine a ratio, which ratio is thereafter used to approximate the scaling factor to be used. The controller of the LP2 (or of the external device) can also perform a similar process to determine the scaling factor for use by the LP2.

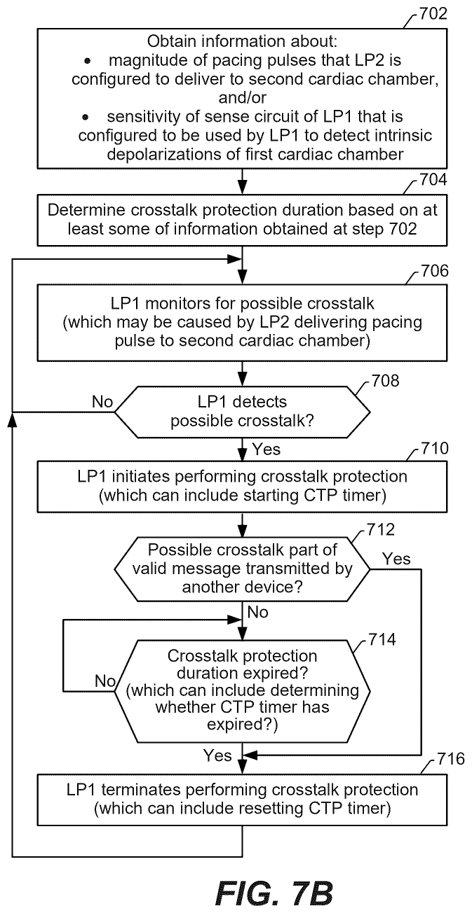

The high level flow diagram of FIG. 7A will now be used to summarize a method according to certain embodiments of the present technology. Such a method is for use with a multi-chamber leadless pacemaker (LP) system including a first leadless pacemaker (LP1) and a second leadless pacemaker (LP2), wherein the LP1 is configured to be implanted in or on a first cardiac chamber of a patient's heart and to deliver pacing pulses to the first cardiac chamber, and the LP2 is configured to be implanted in or on a second cardiac chamber of the patient's heart and to deliver pacing pulses to the second cardiac chamber. For example, the LP1 and the LP2 can be, respectively, the aLP 102a and the vLP 102b. For another example, the LP1 and the LP2 can be, respectively, the vLP 102b and the aLP 102a. Other variations are also possible and with the scope of the embodiments described herein.

Referring to FIG. 7A, step 702 involves obtaining information about a magnitude of the pacing pulses that the LP2 is configured to deliver to the second cardiac chamber, and/or a sensitivity of a sense circuit of the LP1 that is configured to be used by the LP1 to detect intrinsic depolarizations of the first cardiac chamber. Still referring to FIG. 7A, step 704 involves determining a crosstalk protection duration based on at least some of the information obtained at step 702.

In accordance with certain embodiments, when the information obtained at step 702 is information about the magnitude of the pacing pulses that the LP2 is configured to deliver to the second cardiac chamber, step 704 involves determining the crosstalk protection duration based on the magnitude of the pacing pulses such that there is a positive correlation between the magnitude of the pacing pulses and the crosstalk protection duration. The phrase “positive correlation” as used herein to describe a relationship between two different variables means that when one variable increases the other variable also increases, and when one variable decreases the other variable also decreases. Accordingly, in general, the greater the magnitude of the pacing pulses (that the LP2 is configured to deliver to the second cardiac chamber) the longer the crosstalk protection period, and the lower the magnitude of the pacing pulses (that the LP2 is configured to deliver to the second cardiac chamber) the shorter the crosstalk protection period. The magnitude of the pacing pulses can be specified in terms of pulse amplitude and/or pulse width, or pacing pulse charge (P_{PQ}), wherein an example equation for pacing pulse charge (P_{PQ}) was provided above. Example equations for determining the crosstalk protection duration based on the magnitude of pacing pulses and a sense detection threshold of a sense circuit were described above. In an embodiment, where there is a positive correlation between the magnitude of the pacing pulses and the crosstalk protection duration, there can be determination that the crosstalk protection duration has a first duration if the pacing pulses have a first magnitude and has a second duration that is longer than the first duration if the pacing pulses have a second magnitude that is greater than the first magnitude. There can also be a determination that the crosstalk protection duration has a third duration that is shorter than the first duration if the pacing pulses have a third magnitude that is less than the first magnitude. As noted above, and as explained in additional detail below, instead of the sensitivity of the sense circuit being specified by a sense detection threshold of the sense circuit that is configured to be used by the to detect intrinsic depolarizations of the first cardiac chamber, the sensitivity of the sense circuit can alternatively be specified by a gain of the sense circuit that is configured to be used to detect intrinsic depolarizations of the first cardiac chamber, in which case there is a positive correlation between the gain of the sense circuit and the crosstalk protection duration.

In accordance with certain embodiments, when the information obtained at step 702 is information about the sense detection threshold of the sense circuit of the LP1 that is configured to be used by the LP1 to detect intrinsic depolarizations of the first cardiac chamber, step 704 involves determining the crosstalk protection duration based on the sense detection threshold of the sense circuit such that there is a negative correlation between the sense detection threshold of the sense circuit and the crosstalk protection duration. The phrase “negative correlation” as used herein to describe a relationship between two different variables means that when one variable increases the other variable decreases, and when one variable decreases the other variable increases. Accordingly, in general, the greater the sense detection threshold of the sense circuit of the LP1 (that is configured to be used by the LP1 to detect intrinsic depolarizations of the first cardiac chamber) the shorter the crosstalk protection duration, and the lower the sense detection threshold of the sense circuit of the LP1 (that is configured to be used by the LP1 to detect intrinsic depolarizations of the first cardiac chamber) the longer the crosstalk protection duration. In an embodiment, where there is a negative correlation between the sense detection threshold of the sense circuit of the LP1 and the crosstalk protection duration, there can be determination that the crosstalk protection duration has a first duration if the sense detection threshold of the sense circuit of the LP1 has a first magnitude and has a second duration that is shorter than the first duration if the sense detection threshold of the sense circuit of the LP1 has a second magnitude that is greater than the first magnitude. It is noted that where a sense detection threshold is being used by an LP to detect intrinsic depolarizations of the cardiac chamber in (or on) which the LP is implanted, the sense detection threshold can also be referred to more specifically as an intrinsic depolarization detection threshold.

In accordance with certain embodiments, when the information obtained at step 702 is information about the gain of sense circuit of the LP1 that is configured to be used by the LP1 to detect intrinsic depolarizations of the first cardiac chamber, step 704 involves determining the crosstalk protection duration based on the gain of the sense circuit such that there is a positive correlation between the gain of the sense circuit and the crosstalk protection duration. Accordingly, in general, the greater the gain of the sense circuit LP1 (that is configured to be used by the LP1 to detect intrinsic depolarizations of the first cardiac chamber) the longer the crosstalk protection duration, and the lower the gain of the sense circuit LP1 (that is configured to be used by the LP1 to detect intrinsic depolarizations of the first cardiac chamber) the shorter the crosstalk protection duration.

In an embodiment, where there is a positive correlation between the gain of the sense circuit (that is configured to be used to detect intrinsic depolarizations of the first cardiac chamber) and the crosstalk protection duration, there can be determination that the crosstalk protection duration has a first duration if the gain circuit has a first gain and has a second duration that is longer than the first duration if the gain circuit has a second gain that is greater than the first gain. There can also be a determination that the crosstalk protection duration has a third duration that is shorter than the first duration if the gain of the sense circuit (that is configured to be used to detect intrinsic depolarizations of the first cardiac chamber) has a third gain that is less than the first gain.

In accordance with certain embodiments, when the information obtained at step 702 includes information about the magnitude of the pacing pulses that the LP2 is configured to deliver to the second cardiac chamber, and as well as information about the sense detection threshold of the sense circuit of the LP1 that is configured to be used by the LP1 to detect intrinsic depolarizations of the first cardiac chamber, then the crosstalk protection duration determined at step 704 can be based on both the magnitude of the pacing pulses and the sense detection threshold of the sense circuit. In such embodiments there is a positive correlation between the magnitude of the pacing pulses and the crosstalk protection duration. Additionally, in such embodiments there is a negative correlation between the sensitivity and the crosstalk protection duration (if the sensitivity is specified by a sense detection threshold of the gain circuit) or there is a positive correlation between the sensitivity and the crosstalk protection duration (if the sensitivity is instead specified by a gain of the sense circuit). Example equations that can be used in such an embodiment were shown above in EQ2 and EQ3. However, embodiments of the present technology are not limited to using such equations.

Still referring to FIG. 7A, step 706 involves the LP1 monitoring for possible crosstalk that may be caused by the LP2 delivering one of the pacing pulses. Such possible crosstalk monitoring at step 706 can be performing, for example, using one of the techniques described in the description of FIGS. 2-8 of U.S. Pat. No. 10,182,765, titled “Systems and Methods for Classifying Signals of Interest in a Cardiac Rhythm Management Device,” which is incorporated by reference as if set forth fully herein. For example, referring to FIG. 2 that was initially discussed above, assume the sense amplifier 132 (in FIG. 2) of an LP is used to pass signals within a first frequency passband that contains intrinsic cardiac activity (e.g., P-waves and R-waves), then the sense amplifier 132 can also be referred to as an intrinsic activity sense amplifier 132, an intrinsic activation sense circuit, an intrinsic depolarization sense circuit, or the like. In order to monitor for possible crosstalk, the LP1 can also include a separate crosstalk sense circuit that has a passband that is shifted to a significantly higher frequency than the passband of the intrinsic activity sense amplifier 132, to enable the crosstalk sense circuit to be more discriminative of the relatively fast rising and falling edges of crosstalk that may be caused by a pacing pulse produced by another LP. Such a crosstalk sense circuit can be implemented, for example, by the LF receiver 120, or by another sense circuit not specifically shown in FIG. 2. Using such sense circuits, the LP1, and more specifically a controller (e.g., 112) thereof, can classify a sensed signal being evaluated as being possible crosstalk where the sensed signal is passed by the crosstalk sense circuit. This is just one example of circuits and techniques that the LP1 can use to perform possible crosstalk monitoring. In an embodiment, at step 706 the LP1 monitors for possible crosstalk that may be caused by the LP2 delivering one of the pacing pulses, by determining whether a sensed signal is passed by a crosstalk sensing circuit configured to pass signals falling within a crosstalk passband (which contains higher frequencies than those of intrinsic depolarizations). At step 708 possible crosstalk is detected when the sensed signal is passed by the crosstalk sensing circuit, and possible crosstalk is not detected when the sensed signal is not passed by the crosstalk sensing circuit. A lower cutoff frequency of the crosstalk sensing circuit can be, e.g., greater than or equal to 500 Hz. More generally, possible crosstalk can be detected at step 708 if a sensed signal has energy above a threshold within a specified frequency range in which crosstalk, when present, is expected to be. The use of other circuits and/or techniques to monitor for and detect possible crosstalk are also within the scope of the embodiments described herein.

At step 708 there is a determination of whether or not possible crosstalk was detected by the LP1. If the answer to the determination at step 708 is No, then flow returns to step 706. If the answer to the determination at step 708 is Yes, then flow goes to step 710.

Step 710 involves the LP1, in response to detecting the possible crosstalk, initiating performing crosstalk protection. In an embodiment, step 710 (or another step) includes starting a crosstalk protection (CTP) timer that is used to count down to (or count up to) the crosstalk protection duration. In other words, the CTP timer is used to keep track of how long crosstalk protection is performed, so that performance of the crosstalk protection does not exceed the crosstalk protection duration (determined at step 704). At step 714 there is a determination of whether the crosstalk protection duration has expired. In an embodiment, step 714 (or another step) includes determining whether the CTP timer has expired. If the answer to the determination at step 714 is No, then step 714 is repeated until the answer to the determination at step 714 is Yes. When the answer to the determination at step 714 is Yes, i.e., when the crosstalk protection duration has expired, flow goes to step 716. At step 716 the LP1 terminates (i.e., stops) performing the crosstalk protection. In an embodiment, step 716 (or another step) includes resetting or reinitialization of the CTP timer, so that the next time possible crosstalk is protected, the CTP timer is ready to be started again. In an alternative embodiment, the resetting or reinitialization of the CTP timer occurs at step 710, before the CTP timer is started.

There are various manners that the LP1 can perform the crosstalk protection that is initiated at step 710 and is thereafter terminated at step 716 (after expiration of the crosstalk protection duration). For example, the crosstalk protection can be performed by blanking the sense circuit (e.g., 132) that the LP1 uses to monitor for intrinsic depolarizations of the first cardiac chamber, ignoring any possible intrinsic depolarization detected using the sense circuit of the LP1, disabling the sense circuit that the LP1 uses to monitor for intrinsic depolarizations of the first cardiac chamber, ignoring any interrupts produced in response to the sense circuit being used to detect a possible intrinsic depolarization, or disabling generating of interrupts that may be produced in response to the sense circuit detecting a possible intrinsic depolarization. The term interrupts as used herein refers to signals that instruct a controller to stop what it is doing and execute another function instead. A possible intrinsic depolarization can be detected in response to a magnitude of a sense detection threshold of the sense circuit (e.g., 132) being exceeded by a sensed signal. The reason the word “possible” is used in the term or phrase “possible intrinsic depolarization” is because it is possible that an intrinsic depolarization (detected in response to the sense detection threshold of the sense circuit being exceeded by a sensed signal) is not a true intrinsic depolarization, but rather, may be a false positive detection of an intrinsic depolarization. One of skill in the art reading this description will appreciate that crosstalk protection may be performed in other manners while being within the scope of the embodiments described herein.

In certain embodiments, steps 702 and 704 are performed by an external programmer, e.g., 109, which is configured to communicate with the LP1 and the LP2, e.g., using conductive communication, RF communication, and/or inductive communication, but not limited thereto. Where steps 702 and 704 are performed by an external programmer, the external programmer can program the crosstalk protection duration into a memory or one or more registers of the LP1, so that the crosstalk protection duration is available for use by the LP1 when possible crosstalk that may be caused by the LP2 delivering one of the pacing pulses is detected by the LP1. In other embodiments, steps 702 and 704 are performed by the LP1 itself, or more specifically, a controller (e.g., 112) of the LP1. In certain such embodiments, the controller of the LP1 can receive the information it obtains at step 702 from its memory, from an external programmer (e.g., 109), and/or from the other LP.

In accordance with certain embodiments, the controller (e.g., 112) of the LP1 is configured to dynamically adjust the sensitivity of the sense circuit (e.g., 132) of the LP1, which sense circuit the LP1 uses to detect intrinsic depolarizations of the first cardiac chamber in (or on) which the LP1 is implanted. The reason the controller may adjust the sensitivity of the sense circuit may be to reduce intrinsic depolarization detection oversensing (where there are too many false positive intrinsic depolarization detections), or reduce intrinsic depolarization detection undersensing (where there are too many false negative intrinsic depolarization detections), but is not limited thereto.

In certain such embodiments, when the controller (e.g., 112) adjusts the sensitivity of sense circuit, the controller also adjusts the crosstalk protection duration based on the adjusted sensitivity. More specifically, if the sensitivity is specified by a sense detection threshold, the controller of the LP1 determines the crosstalk protection duration such that there is a negative correlation between the sense detection threshold and the crosstalk protection duration, and such that crosstalk protection duration is updated by the controller of the LP1 when the controller of the LP1 adjusts the sense detection threshold of the sense circuit of the LP1. Alternatively, if the sensitivity is specified by a gain of the sense circuit of the LP1 (while the sense detection threshold remains constant), the controller of the LP1 determines the crosstalk protection duration such that there is a positive correlation between the gain and the crosstalk protection duration, and such that crosstalk protection duration is updated by the controller of the LP1 when the controller of the LP1 adjusts the gain of the sense circuit of the LP1.

In accordance with certain embodiments, the controller of the LP2 is configured to dynamically adjust the magnitude of the pacing pulses that the LP2 is configured to deliver to the second cardiac chamber, and when it does, the LP2 informs the controller of LP1 (via an i2i message) of an adjustment made to the magnitude of the pacing pulses. In certain such embodiments, the controller of the LP1 determines the crosstalk protection duration based on the magnitude of the pacing pulses that the LP2 is configured to deliver to the second cardiac chamber, such that the crosstalk protection duration is updated by the controller of the LP1 whenever the controller of the LP1 is informed by the LP2 of the adjustment made to the magnitude of the pacing pulses.

It is possible, that when the LP1 detects possible crosstalk at an instance of step 708, the possible crosstalk that was detected was not actually crosstalk, but was instead a portion of an actual message transmitted to the LP1 by another device. For example, it is possible that the possible crosstalk that was detected was actually a wakeup pulse of an implant-to-implant (i2i) message transmitted by the LP2 (or another implanted device, such as an S-ICD (e.g., 106)), or a wakeup pulse of a programmer-to-implant (p2i) message transmitted by an external programmer (e.g., 109). Where the possible crosstalk that was detected is not actually crosstalk, it would be beneficial if the crosstalk protection (that had been initiated) is terminated as soon as possible, and certainly before the crosstalk protection duration has expired, in order to reduce the probability of the LP1 missing the opportunity to detect an actual intrinsic depolarization of the first cardiac chamber in (or on) which the LP1 is implanted. The high level flow diagram of FIG. 7B illustrates how this can be done.

Referring to FIG. 7B, steps 702-710 are the same as they were in FIG. 7A, and thus need not be described again. As can be appreciated from FIG. 7B, after the LP1 initiates crosstalk protection at step 710, at step 712 the LP1 determines whether the possible crosstalk (which had just been detected at step 708) is actually part of a valid message transmitted by another device. If the answer to the determination at step 712 is Yes, the flow goes to step 716, at which the LP1 terminates performing crosstalk protection. If the answer to the determination at step 712 is No, the flow goes to step 714, and the method proceeds in the manner discussed above with reference to FIG. 7A. In still another embodiment, if the answer to the determination at step 714 is No, flow returns to step 712, instead of returning back to step 714. In accordance with certain embodiments, the LP1 can determine (at step 712) whether possible crosstalk (detected at step 708) is actually part of a valid message transmitted by another device by analyzing the received signal within a message window following when possible crosstalk was detected (at step 708). For example, referring to FIG. 2, because the possible crosstalk may actually be a wakeup pulse of a valid message, in response to the possible crosstalk being detected (e.g., by the LF receiver 120), the HF receiver 122 can be enabled and used to sense a signal within a message window (following when the possible crosstalk was detected), and the signal output from the HF receiver 122 can be provided to the controller 112, and the controller can determine whether a valid message was received by determining whether pulses within the message window are what is expected to be included within a valid message. For example, a message may be considered valid if it is received within the expected time window after the LF wake-up pulse, it includes a valid preamble/marker code, it passes a CRC check (if a CRC was included in the message). Other variations are also possible and within the embodiments described herein.

It should be understood that when an LP (e.g., LP1) performs certain steps or functions, such steps are typically performed by or under the control of the controller (e.g., 112) of the LP, wherein the controller can include a microprocessor and/or a state machine, but not limited thereto. Similarly, when a programmer (e.g., 109) or other external device performs certain steps or functions, such steps are typically performed by or under the control of a controller of the external device.

FIG. 8 shows a block diagram of one embodiment of an IMD (e.g., an LP or ICD) 801 that is implanted into the patient as part of the implantable cardiac system in accordance with certain embodiments herein. Optionally, the IMD 801 may provide full-function cardiac resynchronization therapy. Alternatively, the IMD 801 may be implemented with a reduced set of functions and components. For instance, the IMD may be implemented without ventricular sensing and pacing.

The IMD 801 has a housing 800 to hold the electronic/computing components. Housing 800 (which is often referred to as the “can”, “case”, “encasing”, or “case electrode”) may be programmably selected to act as the return electrode for certain stimulus modes. Housing 800 may further include a connector (not shown) with a plurality of terminals 802, 804, 806, 808, and 810. The terminals may be connected to electrodes that are located in various locations on housing 800 or elsewhere within and about the heart. The IMD 801 includes a programmable microcontroller 820 that controls various operations of the IMD 801, including cardiac monitoring and stimulation therapy. Microcontroller 820 includes a microprocessor (or equivalent control circuitry), RAM and/or ROM memory, logic and timing circuitry, state machine circuitry, and I/O circuitry.

The IMD 801 further includes a first pulse generator 822 that generates stimulation pulses for delivery by one or more electrodes coupled thereto. Pulse generator 822 is controlled by microcontroller 820 via control signal 824. Pulse generator 822 may be coupled to the select electrode(s) via an electrode configuration switch 826, which includes multiple switches for connecting the desired electrodes to the appropriate I/O circuits, thereby facilitating electrode programmability. Switch 826 is controlled by a control signal 828 from microcontroller 820.

In the embodiment of FIG. 8, a single pulse generator 822 is illustrated. Optionally, the IMD may include multiple pulse generators, similar to pulse generator 822, where each pulse generator is coupled to one or more electrodes and controlled by microcontroller 820 to deliver select stimulus pulse(s) to the corresponding one or more electrodes.

Microcontroller 820 is illustrated as including timing control circuitry 832 to control the timing of the stimulation pulses (e.g., pacing rate, atrio-ventricular (AV) delay, atrial interconduction (A-A) delay, or ventricular interconduction (V-V) delay, etc.). Timing control circuitry 832 may also be used for the timing of refractory periods, blanking intervals, noise detection windows, evoked response windows, alert intervals, marker channel timing, and so on. Microcontroller 820 also has an arrhythmia detector 834 for detecting arrhythmia conditions. Microcontroller 820 is also shown as including a crosstalk protection module 836, which can be used for determining a crosstalk protection duration using embodiments of the present technology described herein, and/or can be used for detecting possible crosstalk. Although not shown, the microcontroller 820 may further include other dedicated circuitry and/or firmware/software components that assist in monitoring various conditions of the patient's heart and managing pacing therapies.

The IMD 801 is further equipped with a communication modem (modulator/demodulator) 840 to enable wireless communication with other devices. Modem 840 may include one or more transmitters and two or more receivers as discussed herein in connection with FIG. 2. In one implementation, modem 840 may use low or high frequency modulation. As one example, modem 840 may transmit i2i messages and other signals through conductive communication between a pair of electrodes. The modem 840 can alternatively, or additionally, be used to provide RF communication and/or inductive communication. Modem 840 may be implemented in hardware as part of microcontroller 820, or as software/firmware instructions programmed into and executed by microcontroller 820. Alternatively, modem 840 may reside separately from the microcontroller as a standalone component.

The IMD 801 includes a sensing circuit 844 selectively coupled to one or more electrodes, that perform sensing operations, through switch 826 to detect the presence of cardiac activity in the right chambers of the heart. Sensing circuit 844 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the cardiac signal of interest. The automatic gain control enables the unit to sense low amplitude signal characteristics of atrial fibrillation. Switch 826 determines the sensing polarity of the cardiac signal by selectively closing the appropriate switches. In this way, the clinician may program the sensing polarity independent of the stimulation polarity.

The output of sensing circuit 844 is connected to microcontroller 820 which, in turn, triggers or inhibits the pulse generator 822 in response to the presence or absence of cardiac activity. Sensing circuit 844 receives a control signal 846 from microcontroller 820 for purposes of controlling the gain, threshold, polarization charge removal circuitry (not shown), and the timing of any blocking circuitry (not shown) coupled to the inputs of the sensing circuitry.

In the embodiment of FIG. 8, a single sensing circuit 844 is illustrated. Optionally, the IMD may include multiple sensing circuits, similar to sensing circuit 844, where each sensing circuit is coupled to one or more electrodes and controlled by microcontroller 820 to sense electrical activity detected at the corresponding one or more electrodes. Sensing circuit 844 may operate in a unipolar sensing configuration or in a bipolar sensing configuration.

IMD 801 further includes an analog-to-digital (A/D) data acquisition system (DAS) 850 coupled to one or more electrodes via switch 826 to sample cardiac signals across any pair of desired electrodes. Data acquisition system 850 is configured to acquire intracardiac electrogram signals, convert the raw analog data into digital data, and store the digital data for later processing and/or telemetric transmission to an external device 109 (e.g., a programmer, local transceiver, or a diagnostic system analyzer). Data acquisition system 850 is controlled by a control signal 856 from the microcontroller 820.

Microcontroller 820 is coupled to a memory 860 by a suitable data/address bus. The programmable operating parameters used by microcontroller 820 are stored in memory 860 and used to customize the operation of IMD 801 to suit the needs of a particular patient. Such operating parameters define, for example, pacing pulse amplitude, pulse duration, electrode polarity, rate, sensitivity (e.g., a sense detection threshold or gain), automatic features, arrhythmia detection criteria, and the amplitude, waveshape and vector of each shocking pulse to be delivered to the patient's heart within each respective tier of therapy.

The operating parameters of IMD 801 may be non-invasively programmed into memory 860 through a telemetry circuit 864 in telemetric communication via communication link 866 with external device 109. Telemetry circuit 864 allows intracardiac electrograms and status information relating to the operation of IMD 801 (as contained in microcontroller 820 or memory 860) to be sent to external device 109 through communication link 866.

The IMD 801 can further include magnet detection circuitry (not shown), coupled to microcontroller 820, to detect when a magnet is placed over the unit. A magnet may be used by a clinician to perform various test functions of IMD 801 and/or to signal microcontroller 820 that external device 109 is in place to receive or transmit data to microcontroller 820 through telemetry circuits 864.

The IMD 801 can further include one or more physiological sensors 870. Such sensors are commonly referred to as “rate-responsive” sensors because they are typically used to adjust pacing stimulation rates according to the exercise state of the patient. However, physiological sensor 870 may further be used to detect changes in cardiac output, changes in the physiological condition of the heart, or diurnal changes in activity (e.g., detecting sleep and wake states). Signals generated by physiological sensors 870 are passed to microcontroller 820 for analysis. Microcontroller 820 responds by adjusting the various pacing parameters (such as rate, AV Delay, V-V Delay, etc.) at which the atrial and ventricular pacing pulses are administered. While shown as being included within IMD 801, physiological sensor(s) 870 may be external to IMD 801, yet still be implanted within or carried by the patient. Examples of physiologic sensors might include sensors that, for example, sense respiration rate, pH of blood, ventricular gradient, activity, position/posture, minute ventilation (MV), and so forth.

A battery 872 provides operating power to all of the components in IMD 801. Battery 872 is capable of operating at low current drains for long periods of time, and is capable of providing high-current pulses (for capacitor charging) when the patient requires a shock pulse (e.g., in excess of 2 A, at voltages above 2 V, for periods of 10 seconds or more). Battery 872 also desirably has a predictable discharge characteristic so that elective replacement time can be detected. As one example, IMD 801 employs lithium/silver vanadium oxide batteries.

IMD 801 further includes an impedance measuring circuit 874, which can be used for many things, including: lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves; and so forth. Impedance measuring circuit 874 is coupled to switch 826 so that any desired electrode may be used. In this embodiment IMD 801 further includes a shocking circuit 880 coupled to microcontroller 820 by a data/address bus 882.

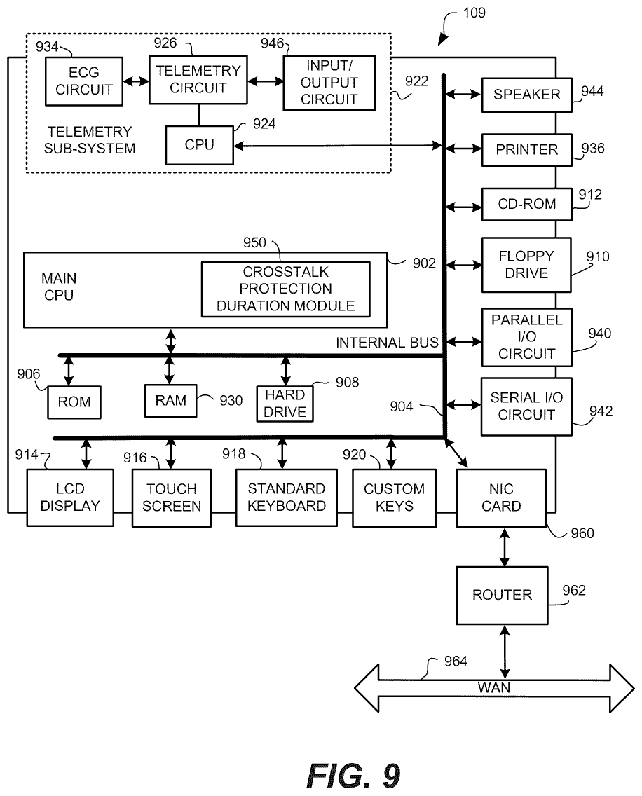

FIG. 9 illustrates example components of an example external device 109 for use in communicating with and/or programming the LPs 102, or other type of IMD. More generally, the external device 109 may permit a physician or other authorized user to program the operation of the LPs. Further, the external device 109 may be capable of causing the LPs 102 to perform functions necessary to complete certain methods of the present technology.

Now, considering the components of the external device 109 by reference to FIG. 9, operations of the external device 109 can be controlled by a CPU 902, which may be a generally programmable microprocessor or microcontroller or may be a dedicated processing device such as an Application Specific Integrated Circuit (ASIC) or the like.

Software instructions to be performed by the CPU can be accessed via an internal bus 904 from a Read Only Memory (ROM) 906 and Random Access Memory (RAM) 930.

Additional software may be accessed from a hard drive 908, floppy drive 910, and CD ROM drive 912, or other suitable permanent mass storage device. Depending upon the specific implementation, a Basic Input Output System (BIOS) is retrieved from the ROM by CPU at power up. Based upon instructions provided in the BIOS, the CPU “boots up” the overall system in accordance with well-established computer processing techniques.

Once operating, the CPU displays a menu of programming options to the user via an LCD display 914 or another suitable computer display device. To this end, the CPU may, for example, display a menu of specific programming parameters of the LP(s) 102 to be programmed or may display a menu of types of diagnostic data to be retrieved and displayed. In response thereto, the physician enters various commands via either a touch screen 916 overlaid on LCD display 914 or through a standard keyboard 918 supplemented by additional custom keys 920, such as an emergency VVI (EVVI) key. The EVVI key sets the LP(s) 102 to a safe WVI mode with high pacing outputs. This ensures life-sustaining pacing operation in nearly all situations but by no means is it desirable to leave cardiac stimulation device 100 in the EVVI mode at all times.

Typically, the physician initially controls the external device 109 to retrieve data stored within one or more of the LPs 102. To this end, CPU 902 transmits appropriate signals to a telemetry circuit 922, which provides components for directly interfacing with the LP(s) 102. The telemetry subsystem 922 can include its own separate CPU 924 for coordinating the operations of the telemetry subsystem 922. The main CPU 902 of the external device 109 communicates with telemetry subsystem CPU 924 via internal bus 904. The telemetry subsystem 922 additionally includes a telemetry circuit 926 for communicating with the LP(s). The telemetry subsystem 922 may utilize one or more types of communication technology to communicate with the LP(s), such as, but not limited to, conductive communication, RF communication, or inductive communication. Patient and device diagnostic data stored within the LP(s) 102 can be transferred to the external device 109. Further, the LP(s) 102 can be instructed to perform an electrode algorithms of the present invention, details of which are provided above. The CPU 902 can include a crosstalk protection duration module 950 that is used to determine a crosstalk protection duration, in accordance with certain embodiments of the present technology.

The external device 109 can also include a Network Interface Card (“NIC”) 960 to permit transmission of data to and from other computer systems via a router 962 and Wide Area Network (“WAN”) 964. Alternatively, the external device 109 might include a modem for communication via the Public Switched Telephone Network (PSTN).

Depending upon the implementation, the modem may be connected directly to internal bus 904 and may be connected to the internal bus via either a parallel port 940 or a serial port 942. Data transmitted from other computer systems may include, for example, data regarding medication prescribed, administered, or sold to the patient.

The external device 109 receives data from the LP(s) 102, including parameters representative of the current programming state of the LP(s) 102. The external device 109 can also receive electrograms (EGMs), samples thereof, and/or date indicative thereof from the LP(s) 102. Under the control of the physician, external device 109 displays the current programming parameters and permits the physician to reprogram the parameters. To this end, the physician enters appropriate commands via any of the aforementioned input devices and, under control of the CPU 902, the programming commands are converted to specific programming parameters for transmission to the LP(S) 102 to thereby reprogram the LP(s) 102. Prior to reprogramming specific parameters, the physician may control the external programmer to display any or all of the data retrieved from the LP(s) 102, including displays of ECGs, displays of electrodes that are candidates as cathodes and/or anodes, and statistical patient information. Any or all of the information displayed by external device 109 may also be printed using a printer 936.

A speaker 944 is included for providing audible tones to the user, such as a warning beep in the event improper input is provided by the physician. Telemetry subsystem 922 may additionally include an input/output circuit 946 which can control the transmission of analog output signals, such as ECG signals output to an ECG machine or chart recorder. Other peripheral devices may be connected to the external device 109 via parallel port 940 or a serial port 942 as well. Although one of each is shown, a plurality of Input Output (IO) ports might be provided.

With the external device 109 configured as shown, a physician or other authorized user can retrieve, process, and display a wide range of information received from the LP(s) 102 and reprogram the LP(s) 102, if needed. The descriptions provided herein with respect to FIG. 9 are intended merely to provide an overview of the operation of the example external device 109 and are not intended to describe in detail every feature of the hardware and software of the device and are not intended to provide an exhaustive list of the functions performed by the device. In accordance with an embodiment, a controller (e.g., 902) of the external device 109 is configured to obtain information about one or more of: a sensitivity of a sense circuit of a first LP (LP1) that is configured to be used by the LP1 to detect intrinsic depolarizations of a first cardiac chamber in or one which the LP1 is implanted, or a magnitude of the pacing pulses that a second LP (LP2) is configured to deliver to a second cardiac chamber in or on which the LP2 is implanted. Additionally, the controller (e.g., 902) of the external device 109 is configured to determine a crosstalk protection duration based on at least some of the information, wherein the crosstalk protection duration after being determined is used by the LP1 to perform crosstalk protection for the crosstalk protection duration in response to the LP1 detecting possible crosstalk that may be caused by the LP2 delivering one of the pacing pulses.

An aspect of the embodiments relates to a system for use with or including a first leadless pacemaker (LP1) and a second leadless pacemaker (LP2), wherein the LP1 is configured to be implanted in or on a first cardiac chamber of a patient's heart and to deliver pacing pulses to the first cardiac chamber, and the LP2 is configured to be implanted in or on a second cardiac chamber of the patient's heart and to deliver pacing pulses to the second cardiac chamber. The system comprises a controller configured to obtain information about one or more of: a magnitude of the pacing pulses that the LP2 is configured to deliver to the second cardiac chamber; or a sensitivity of a sense circuit of the LP1 that is configured to be used by the LP1 to detect intrinsic depolarizations of the first cardiac chamber. The controller is also configured to determine a crosstalk protection duration based on at least some of the information, wherein the crosstalk protection duration after being determined is used by the LP1 to perform crosstalk protection for the crosstalk protection duration in response to the LP1 detecting possible crosstalk that may be caused by the LP2 delivering one of the pacing pulses.

An aspect of the embodiments relates to a system comprising the first leadless pacemaker (LP1) configured to be implanted in or on the first cardiac chamber of the patient's heart and comprising: a pulse generator configured to deliver pacing pulses to the first cardiac chamber; and a sense circuit configured to detect intrinsic depolarizations of the first cardiac chamber. The system also comprises the second leadless pacemaker (LP2) configured to be implanted in or on the second cardiac chamber of the patient's heart and comprising: a pulse generator configured to deliver pacing pulses to the second cardiac chamber; and a sense circuit configured to detect intrinsic depolarizations of the second cardiac chamber. The system also comprises the controller configured to: obtain information about one or more of a magnitude of the pacing pulses that the pulse generator of the LP2 is configured to deliver to the second cardiac chamber, or a sensitivity of the sense circuit of the LP1 that is configured to be used by the LP1 to detect intrinsic depolarizations of the first cardiac chamber; and determine a crosstalk protection duration based on at least some of the information. In other words, the controller can be configured to determine a length of the crosstalk protection duration based on the information about the magnitude of the pacing pulses that the pulse generator of the LP2 is configured to deliver to the second cardiac chamber, and/or based on the information about the sensitivity of the sense circuit of the LP1 that is configured to be used by the LP1 to detect intrinsic depolarizations of the first cardiac chamber. In certain such embodiments, the LP1 is configured to, in response to the LP1 detecting possible crosstalk that may be caused by the pulse generator of the LP2 delivering one of the pacing pulses, perform crosstalk protection for the crosstalk protection duration.

In an embodiment, the controller is configured to: obtain information about the magnitude of the pacing pulses that the LP2 is configured to deliver to the second cardiac chamber; and determine the crosstalk protection duration based on the magnitude of the pacing pulses such that there is a positive correlation between the magnitude of the pacing pulses and the crosstalk protection duration. In an embodiment, where the controller is configured to determine the crosstalk protection duration based on the magnitude of the pacing pulses, such that there is a positive correlation between the magnitude of the pacing pulses and the crosstalk protection duration, the controller is configured to determine that the crosstalk protection duration has a first duration if the pacing pulses have a first magnitude and have a second duration that is longer than the first duration if the pacing pulses has a second magnitude that is greater than the first magnitude.

In an embodiment, the controller is configured to: obtain information about the magnitude of the pacing pulses that the LP2 is configured to deliver to the second cardiac chamber by obtaining information about at least one of a pulse amplitude or a pulse width of the pacing pulses that the LP2 is configured to deliver to the second cardiac chamber.

In an embodiment, the controller is configured to obtain information about the sensitivity of the sense circuit of the LP1. In certain such embodiments, the sensitivity of the sense circuit of the LP1 is specified by a sense detection threshold of the sense circuit of the LP1 that is configured to be used by the LP1 to detect intrinsic depolarizations of the first cardiac chamber, and the controller is configured to determine the crosstalk protection duration based on the sense detection threshold of the sense circuit such that there is a negative correlation between the sense detection threshold of the sense circuit and the crosstalk protection duration. In an embodiment, where the controller is configured to determine the crosstalk protection duration based on the sense detection threshold of the sense circuit of the LP1, such that there is a negative correlation between the sense detection threshold of the sense circuit of the LP1 and the crosstalk protection duration, the controller is configured to determine that the crosstalk protection duration has a first duration if the sense detection threshold of the sense circuit of the LP1 has a first magnitude and has a second duration that is shorter than the first duration if the sense detection threshold of the sense circuit of the LP1 has a second magnitude that is greater than the first magnitude. Alternatively, the sensitivity of the sense circuit of the LP1 is specified by a gain of the sense circuit of the LP1 that is configured to be used by the LP1 to detect intrinsic depolarizations of the first cardiac chamber and the controller is configured to determine the crosstalk protection duration based on the gain of the sense circuit such that there is a positive correlation between the gain of the sense circuit and the crosstalk protection duration. In an embodiment, where the controller is configured to determine the crosstalk protection duration based on the gain of the sense circuit of the LP1 (that is configured to be used to detect intrinsic depolarizations of the first cardiac chamber), such that there is a positive correlation between the gain of the sense circuit (that is configured to be used to detect intrinsic depolarizations of the first cardiac chamber) and the crosstalk protection duration, the controller is configured to determine that the crosstalk protection duration has a first duration if the gain circuit has a first gain and has a second duration that is longer than the first duration if the gain circuit has a second gain that is greater than the first gain. The controller can also be configured to determine that the crosstalk protection duration has a third duration that is shorter than the first duration if the gain of the sense circuit (that is configured to be used to detect intrinsic depolarizations of the first cardiac chamber) has a third gain that is less than the first gain.

In an embodiment, the controller is configured to: obtain information about the magnitude of the pacing pulses that the LP2 is configured to deliver to the second cardiac chamber and obtain information about the sensitivity of the sense circuit of the LP1 that is configured to be used by the LP1 to detect intrinsic depolarizations of the first cardiac chamber; and determine the crosstalk protection duration based on the magnitude of the pacing pulses and the sensitivity of the sense circuit.

In an embodiment, the controller is configured to: determine the crosstalk protection duration also based on a scaling factor that is related to at least one of a distance between the LP1 and the LP2 or an angle of the LP1 and the LP2 relative to one another.

In an embodiment, the system comprises a portion of the LP1 that includes the controller, wherein the controller of the LP1 is configured to: monitor for and detect possible crosstalk that may be caused by the LP2 delivering one of the pacing pulses; and initiate performance of crosstalk protection for the crosstalk protection duration in response to detecting possible crosstalk that may be caused by the LP2 delivering one of the pacing pulses.

In an embodiment, the controller of the LP1 is configured to: dynamically adjust the sensitivity of the sense circuit of the LP1; and determine the crosstalk protection duration based on the sensitivity such that the crosstalk protection duration is updated by the controller of the LP1 when the controller of the LP1 adjusts the sensitivity of the sense circuit of the LP1.

In an embodiment, a controller of the LP2 is configured to dynamically adjust the magnitude of the pacing pulses that the LP2 is configured to deliver to the second cardiac chamber, and to inform the controller of LP1 of adjustments made to the magnitude of the pacing pulses; and the controller of the LP1 is configured to determine the crosstalk protection duration based on the magnitude of the pacing pulses that the LP2 is configured to deliver to the second cardiac chamber, such that there is a positive correlation between the magnitude of the pacing pulses and the crosstalk protection duration, and such that the crosstalk protection duration is updated by the controller of the LP1 in response to the controller of the LP1 being informed of the adjustments made to the magnitude of the pacing pulses.

In an embodiment, the system comprises a non-implantable programmer that includes the controller (that is configured to determine the crosstalk protection duration) and is configured to communicate with the LP1 and the LP2 (directly or through an intermediary such as another IMD), and wherein the non-implantable programmer is configured to program the crosstalk protection duration into a memory or one or more registers of the LP1 so that the crosstalk protection duration is available for use by the LP1 when possible crosstalk that may be caused by the LP2 delivering one of the pacing pulses is detected by the LP1.

In an embodiment, the system comprises a portion of the LP1 that includes the controller, wherein the controller of the LP1 is configured to provide crosstalk protection for the crosstalk protection duration by causing at least one of: blanking of the sense circuit of the LP1 for the crosstalk protection duration; ignoring of any possible intrinsic depolarization detected using the sense circuit of the LP1 during the crosstalk protection duration; disabling of the sense circuit of the LP1 for the crosstalk protection duration; ignoring of any interrupts produced in response to the sense circuit being used to detect a possible intrinsic depolarization during the crosstalk protection duration; and disabling of generating of interrupts that may be produced in response to the sense circuit detecting an intrinsic depolarization during the crosstalk protection duration.

In an embodiment, the system comprises a portion of the LP1 that includes the controller, and wherein the controller of the LP1 is configured to: determine whether the possible crosstalk that was detected is part of a valid message transmitted by another device; and terminate the crosstalk protection for a remainder of the crosstalk protection duration in response to determining that the possible crosstalk that was detected is part of the valid message transmitted by another device.

In an embodiment, the LP1 is configured to provide crosstalk protection for the crosstalk protection duration by causing at least one of the following: blanking of the sense circuit of the LP1 for the crosstalk protection duration; ignoring of any possible intrinsic depolarization detected using the sense circuit of the LP1 during the crosstalk protection duration; disabling of the sense circuit of the LP1 for the crosstalk protection duration; ignoring of any interrupts produced in response to the sense circuit being used to detect a possible intrinsic depolarization during the crosstalk protection duration; or disabling of generating of interrupts that may be produced in response to the sense circuit detecting an intrinsic depolarization, during the crosstalk protection duration.

Another aspect of the embodiments relates to a leadless pacemaker configured to communicate with another leadless pacemaker, wherein the leadless pacemaker is configured to be implanted in or on a first cardiac chamber of a patient's heart and to deliver pacing pulses to the first cardiac chamber, and the other leadless pacemaker is configured to be implanted in or on a second cardiac chamber of the patient's heart and to deliver pacing pulses to the second cardiac chamber. The leadless pacemaker includes a sense circuit and a controller. The sense circuit is configured to be used to detect intrinsic depolarizations of the first cardiac chamber. The controller is configured to obtain information about one or more of: a magnitude of the pacing pulses that the other leadless pacemaker is configured to deliver to the second cardiac chamber, or a sensitivity of the sense circuit. The controller is also configured to: determine a crosstalk protection duration based on at least some of the information; monitor for possible crosstalk that may be caused by the other leadless pacemaker delivering one of the pacing pulses; and perform crosstalk protection for the crosstalk protection duration in response to detecting the possible crosstalk that may be caused by the other leadless pacemaker delivering one of the pacing pulses.

In an embodiment, the controller is configured to obtain information about the magnitude of the pacing pulses that the other leadless pacemaker is configured to deliver to the second cardiac chamber, and based thereon determine the crosstalk protection duration such that there is a positive correlation between the magnitude of the pacing pulses and the crosstalk protection duration.

In an embodiment, the controller is configured to: obtain information about a sense detection threshold of the sense circuit and determine the crosstalk protection duration based on the sense detection threshold of the sense circuit such that there is a negative correlation between the sense detection threshold of the sense circuit and the crosstalk protection duration; or obtain information about a gain of the sense circuit and determine the crosstalk protection duration based on the gain of the sense circuit such that there is a positive correlation between the gain of the sense circuit and the crosstalk protection duration.

In an embodiment, the controller is configured to: obtain information about the magnitude of the pacing pulses that the other leadless pacemaker is configured to deliver to the second cardiac chamber; obtain information about the sensitivity of the sense circuit; and determine the crosstalk protection duration based on the magnitude of the pacing pulses and the sensitivity of the sense circuit.

In an embodiment, the controller is configured to determine the crosstalk protection duration also based on a scaling factor that is related to at least one of a distance between the leadless pacemaker and the other leadless pacemaker, or an angle of the leadless pacemaker and the other leadless pacemaker relative to one another.

In an embodiment, the controller is configured to: dynamically adjust the sensitivity of the sense circuit; and determine the crosstalk protection duration based on the sensitivity of the sense circuit, such that the crosstalk protection duration is updated when the sensitivity of the sense circuit is adjusted.

In an embodiment, the controller is configured to: determine the crosstalk protection duration based on the magnitude of the pacing pulses that the other leadless pacemaker is configured to deliver to the second cardiac chamber, such that there is a positive correlation between the magnitude of the pacing pulses and the crosstalk protection duration, and such that the crosstalk protection duration is updated in response to being informed of the adjustments made to the magnitude of the pacing pulses that the other leadless pacemaker is configured to deliver to the second cardiac chamber.

In an embodiment, the controller is configured to provide crosstalk protection, for the crosstalk protection duration, by causing at least one of: blanking of the sense circuit for the crosstalk protection duration; ignoring of any possible intrinsic depolarization detected using the sense circuit during the crosstalk protection duration; disabling of the sense circuit for the crosstalk protection duration; ignoring of any interrupts produced in response to the sense circuit being used to detect a possible intrinsic depolarization during the crosstalk protection duration; or disabling of generating of interrupts that may be produced in response to the sense circuit detecting an intrinsic depolarization during the crosstalk protection duration.

In an embodiment, the controller is configured to: determine whether the possible crosstalk that was detected is part of a valid message transmitted by another device; and terminate the crosstalk protection for a remainder of the crosstalk protection duration, in response to determining that the possible crosstalk that was detected is part of the valid message transmitted by another device.

Another aspect of the embodiments relates to a method for use with a dual chamber leadless pacemaker (LP) system including a first leadless pacemaker (LP1) and a second leadless pacemaker (LP2), wherein the LP1 is configured to be implanted in or on a first cardiac chamber of a patient's heart and to deliver pacing pulses to the first cardiac chamber, and the LP2 is configured to be implanted in or on a second cardiac chamber of the patient's heart and to deliver pacing pulses to the second cardiac chamber. The method comprises obtaining information about one or more of: a magnitude of the pacing pulses that the LP2 is configured to deliver to the second cardiac chamber; or a sensitivity of a sense circuit of the LP1 that is configured to be used by the LP1 to detect intrinsic depolarizations of the first cardiac chamber. The method also includes determining a crosstalk protection duration based on at least some of the information. The method additionally includes the LP1 monitoring for and detecting possible crosstalk that may by caused by the LP2 delivering one of the pacing pulses, and the LP1 in response to detecting the possible crosstalk that may by caused by the LP2 delivering one of the pacing pulses initiating providing crosstalk protection for the crosstalk protection duration.

In an embodiment, the obtaining information comprises obtaining information about the magnitude of the pacing pulses that the LP2 is configured to deliver to the second cardiac chamber; and the determining the crosstalk protection duration is based on the magnitude of the pacing pulses such that there is a positive correlation between the magnitude of the pacing pulses and the crosstalk protection duration. In an embodiment, the obtaining information about the magnitude of the pacing pulses that the LP2 is configured to deliver to the second cardiac chamber comprises obtaining information about at least one of a pulse amplitude or a pulse width of the pacing pulses that the LP2 is configured to deliver to the second cardiac chamber.

In an embodiment, the obtaining information comprises obtaining information about the sensitivity of the sense circuit of the LP1. In certain such embodiments, the sensitivity of the sense circuit of the LP1 is specified by a sense detection threshold of the sense circuit of the LP1 that is configured to be used by the LP1 to detect intrinsic depolarizations of the first cardiac chamber, and the determining the crosstalk protection duration is based on the sense detection threshold of the sense circuit such that there is a negative correlation between the sense detection threshold of the sense circuit and the crosstalk protection duration. In an embodiment, where the controller is configured to determine the crosstalk protection duration based on the sense detection threshold of the sense circuit of the LP1, such that there is a negative correlation between the sense detection threshold of the sense circuit of the LP1 and the crosstalk protection duration, the controller is configured to determine that the crosstalk protection duration has a first duration if the sense detection threshold of the sense circuit of the LP1 has a first magnitude and has a second duration that is shorter than the first duration if the sense detection threshold of the sense circuit of the LP1 has a second magnitude that is greater than the first magnitude. Alternatively, the sensitivity of the sense circuit of the LP1 is specified by a gain of the sense circuit of the LP1 that is configured to be used by the LP1 to detect intrinsic depolarizations of the first cardiac chamber, and the determining the crosstalk protection duration is based on the gain of the sense circuit such that there is a positive correlation between the gain of the sense circuit and the crosstalk protection duration.

In an embodiment, the obtaining information comprises obtaining information about the magnitude of the pacing pulses that the LP2 is configured to deliver to the second cardiac chamber and obtaining information about the sensitivity of the sense circuit of the LP1 that is configured to be used by the LP1 to detect intrinsic depolarizations of the first cardiac chamber; and the determining the crosstalk protection duration is based on the magnitude of the pacing pulses and the sensitivity of the sense circuit.

In an embodiment, the determining the crosstalk protection duration is also based on a scaling factor that is related to at least one of a distance between the LP1 and the LP2 or an angle of the LP1 and the LP2 relative to one another.

In an embodiment, the determining the crosstalk protection duration is performed by a controller of the LP1.

In an embodiment, the controller of the LP1 is configured to dynamically adjust the sensitivity of the sense circuit of the LP1; and the determining the crosstalk protection duration is based on the sensitivity such that crosstalk protection duration is updated by the controller of the LP1 when the controller of the LP1 adjusts the sensitivity of the sense circuit of the LP1.

In an embodiment, a controller of the LP2 is configured to dynamically adjust the magnitude of the pacing pulses that the LP2 is configured to deliver to the second cardiac chamber and to inform the controller of LP1 of adjustments made to the magnitude of the pacing pulses; and the determining the crosstalk protection duration is based on the magnitude of the pacing pulses that the LP2 is configured to deliver to the second cardiac chamber, such that there is a positive correlation between the magnitude of the pacing pulses and the crosstalk protection duration, and such that the crosstalk protection duration is updated by the controller of the LP1 in response to the controller of the LP1 being informed by the LP2 of the adjustments made to the magnitude of the pacing pulses.

In an embodiment, the obtaining the information and the determining the crosstalk protection duration are performed by a non-implantable programmer; and the method further comprises the non-implantable programmer programming the crosstalk protection duration into a memory or one or more registers of the LP1, so that the crosstalk protection duration is available for use by the LP1 when possible crosstalk (that may be caused by the LP2 delivering one of the pacing pulses) is detected by the LP1.

In an embodiment, the providing crosstalk protection for the crosstalk protection duration comprises at least one of the following: blanking the sense circuit of the LP1 for the crosstalk protection duration; ignoring any possible intrinsic depolarization detected using the sense circuit of the LP1 during the crosstalk protection duration; disabling the sense circuit of the LP1 for the crosstalk protection duration; ignoring any interrupts produced in response to the sense circuit being used to detect a possible intrinsic depolarization during the crosstalk protection duration; or disabling generating of interrupts that may be produced in response to the sense circuit detecting an intrinsic depolarization during the crosstalk protection duration.

In an embodiment, the method further comprises determining that the possible crosstalk that was detected is part of a valid message transmitted by another device and in response thereto terminating the crosstalk protection for a remainder of the crosstalk protection duration.

Embodiments of the present technology have been described above with the aid of functional building blocks illustrating the performance of specified functions and relationships thereof. The boundaries of these functional building blocks have often been defined herein for the convenience of the description. Alternate boundaries can be defined so long as the specified functions and relationships thereof are appropriately performed. Any such alternate boundaries are thus within the scope and spirit of the claimed invention. For example, it would be possible to combine or separate some of the steps shown in FIGS. 7A and 7B. It would also be possible to reorder some of the steps shown in FIGS. 7A and 7B. For another example, it is possible to change the boundaries of some of the blocks shown in FIGS. 2, 8, and 9.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, it is noted that the term “based on” as used herein, unless stated otherwise, should be interpreted as meaning based at least in part on, meaning there can be one or more additional factors upon which a decision or the like is made. For example, if a decision is based on the results of a comparison, that decision can also be based on one or more other factors in addition to being based on results of the comparison.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the embodiments of the present technology without departing from its scope. While the dimensions, types of materials and coatings described herein are intended to define the parameters of the embodiments of the present technology, they are by no means limiting and are exemplary embodiments. Many other embodiments will be apparent to those of skill in the art upon reviewing the above description. The scope of the embodiments of the present technology should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112(f), unless and until such claim limitations expressly use the phrase “means for” followed by a statement of function void of further structure.

What is claimed is:

1. A system for use with or including a first leadless pacemaker (LP1) and a second leadless pacemaker (LP2), wherein the LP1 is configured to be implanted in or on a first cardiac chamber of a patient's heart and to deliver pacing pulses to the first cardiac chamber, and wherein the LP2 is configured to be implanted in or on a second cardiac chamber of the patient's heart and to deliver pacing pulses to the second cardiac chamber, the system comprising:
a controller configured to
obtain information about one or more of
a magnitude of the pacing pulses that the LP2 is configured to deliver to the second cardiac chamber, or
a sensitivity of a sense circuit of the LP1 that is configured to be used by the LP1 to detect intrinsic depolarizations of the first cardiac chamber; and
determine a crosstalk protection duration based on at least some of the information;
wherein the crosstalk protection duration after being determined is used by the LP1 to perform crosstalk protection for the crosstalk protection duration in response to the LP1 detecting possible crosstalk that may be caused by the LP2 delivering one of the pacing pulses.

2. The system of claim 1, wherein the controller is configured to:
obtain information about the magnitude of the pacing pulses that the LP2 is configured to deliver to the second cardiac chamber by obtaining information about at least one of a pulse amplitude or a pulse width of the pacing pulses that the LP2 is configured to deliver to the second cardiac chamber; and
determine the crosstalk protection duration based on the magnitude of the pacing pulses such that there is a positive correlation between the magnitude of the pacing pulses and the crosstalk protection duration.

3. The system of claim 1, wherein the controller is configured to obtain information about the sensitivity of the sense circuit of the LP1, and wherein:
the sensitivity of the sense circuit of the LP1 is specified by a sense detection threshold of the sense circuit of the LP1 that is configured to be used by the LP1 to detect intrinsic depolarizations of the first cardiac chamber, and the controller is configured to determine the crosstalk protection duration based on the sense detection threshold of the sense circuit of the LP1 such that there is a negative correlation between the sense detection threshold of the sense circuit of the LP1 and the crosstalk protection duration; or
the sensitivity of the sense circuit of the LP1 is specified by a gain of the sense circuit of the LP1 that is configured to be used by the LP1 to detect intrinsic depolarizations of the first cardiac chamber, and the controller is configured to determine the crosstalk protection duration based on the gain of the sense circuit of the LP1 such that there is a positive correlation between the gain of the sense circuit of the LP1 and the crosstalk protection duration.

4. The system of claim 1, wherein the controller is configured to:
obtain information about the magnitude of the pacing pulses that the LP2 is configured to deliver to the second cardiac chamber, and obtain information about the sensitivity of the sense circuit of the LP1 that is configured to be used by the LP1 to detect intrinsic depolarizations of the first cardiac chamber; and
determine the crosstalk protection duration based on both the magnitude of the pacing pulses that the LP2 is configured to deliver to the second cardiac chamber and the sensitivity of the sense circuit of the LP1.

5. The system of claim 1, wherein the system comprises a portion of the LP1 that includes the controller, and wherein the controller of the LP1 is configured to:
monitor for and detect possible crosstalk that may be caused by the LP2 delivering one of the pacing pulses; and
initiate performance of crosstalk protection for the crosstalk protection duration in response to detecting possible crosstalk that may be caused by the LP2 delivering one of the pacing pulses.

6. The system of claim 5, wherein the controller of the LP1 is configured to dynamically adjust the sensitivity of the sense circuit of the LP1 and update the crosstalk protection duration in response to the sensitivity of the sense circuit of the LP1 being adjusted; and wherein:
the sensitivity of the sense circuit of the LP1 is specified by a sense detection threshold of the sense circuit of the LP1 that is configured to be used by the LP1 to detect intrinsic depolarizations of the first cardiac chamber, and the controller of the LP1 is configured to determine the crosstalk protection duration based on the sense detection threshold of the sense circuit of the LP1 such that there is a negative correlation between the sense detection threshold of the sense circuit of the LP1 and the crosstalk protection duration; or
the sensitivity of the sense circuit of the LP1 is specified by a gain of the sense circuit of the LP1 that is configured to be used by the LP1 to detect intrinsic depolarizations of the first cardiac chamber, and the controller of the LP1 is configured to determine the crosstalk protection duration based on the gain of the sense circuit of the LP1 such that there is a positive correlation between the gain of the sense circuit of the LP1 and the crosstalk protection duration.

7. The system of claim 5, wherein:
a controller of the LP2 is configured to dynamically adjust the magnitude of the pacing pulses that the LP2 is configured to deliver to the second cardiac chamber and to inform the controller of LP1 of adjustments made to the magnitude of the pacing pulses; and
the controller of the LP1 is configured to determine the crosstalk protection duration based on the magnitude of the pacing pulses that the LP2 is configured to deliver to the second cardiac chamber, such that there is a positive correlation between the magnitude of the pacing pulses and the crosstalk protection duration and such that the crosstalk protection duration is updated by the controller of the LP1 in response to the controller of the LP1 being informed of the adjustments made to the magnitude of the pacing pulses.

8. The system of claim 1, wherein the system comprises a portion of the LP1 that includes the controller, and wherein the controller of the LP1 is configured to provide crosstalk protection, for the crosstalk protection duration, by causing at least one of the following:
blanking of the sense circuit of the LP1 for the crosstalk protection duration;
ignoring of any possible intrinsic depolarization detected using the sense circuit of the LP1 during the crosstalk protection duration;
disabling of the sense circuit of the LP1 for the crosstalk protection duration;
ignoring of any interrupts produced in response to the sense circuit being used to detect a possible intrinsic depolarization during the crosstalk protection duration; or
disabling of generating of interrupts that may be produced in response to the sense circuit detecting an intrinsic depolarization during the crosstalk protection duration.

9. The system of claim 1, wherein the system comprises a portion of the LP1 that includes the controller, and wherein the controller of the LP1 is configured to:
determine whether the possible crosstalk that was detected is part of a valid message transmitted by another device; and
terminate the crosstalk protection for a remainder of the crosstalk protection duration in response to determining that the possible crosstalk that was detected is part of the valid message transmitted by another device.

10. The system of claim 1, wherein the system comprises a non-implantable programmer that includes the controller and is configured to communicate with the LP1 and the LP2, and wherein the non-implantable programmer is configured to program the crosstalk protection duration into a memory or one or more registers of the LP1 so that the crosstalk protection duration is available for use by the LP1 when possible crosstalk that may be caused by the LP2 delivering one of the pacing pulses is detected by the LP1.

11. A medical device comprising or for use with a leadless pacemaker configured to be implanted in or on a first cardiac chamber of a patient's heart and configured to communicate with another leadless pacemaker configured to be implanted in or on a second cardiac chamber of the patient's heart and to deliver pacing pulses to the second cardiac chamber, medical device comprising:
a controller configured to
obtain information about one or more of
a magnitude of the pacing pulses that the other leadless pacemaker is configured to deliver to the second cardiac chamber, or
a sensitivity of a sense circuit of the leadless pacemaker that is configured to be used by the leadless pacemaker to detect intrinsic depolarizations of the first cardiac chamber;
determine a crosstalk protection duration based on at least some of the information; and
cause the leadless pacemaker to perform crosstalk protection for the crosstalk protection duration in response to the leadless pacemaker detecting possible crosstalk that may be caused by the other leadless pacemaker delivering one of the pacing pulses to the second cardiac chamber.

12. The medical device of claim 11, wherein:
the medical device comprises the leadless pacemaker;
the leadless pacemaker comprises the controller and the sense circuit configured to be used to detect intrinsic depolarizations of the first cardiac chamber;
the controller of the leadless pacemaker is further configured to:
monitor for possible crosstalk that may be caused by the other leadless pacemaker delivering one of the pacing pulses to the second cardiac chamber; and
perform crosstalk protection for the crosstalk protection duration in response to detecting the possible crosstalk that may be caused by the other leadless pacemaker delivering one of the pacing pulses to the second cardiac chamber.

13. The medical device of claim 11, wherein:
the medical device comprises a non-implantable programmer that includes the controller;
the non-implantable programmer is configured to communicate with the leadless pacemaker and the other leadless pacemaker; and
the non-implantable programmer is configured to program the crosstalk protection duration into a memory or one or more registers of the leadless pacemaker so that the crosstalk protection duration is available for use by the leadless pacemaker when possible crosstalk that may be caused by the other leadless pacemaker delivering one of the pacing pulses to the second cardiac chamber is detected by the leadless pacemaker.

14. The medical device of claim 11, wherein the controller is configured to:
obtain information about the magnitude of the pacing pulses that the other leadless pacemaker is configured to deliver to the second cardiac chamber and based thereon, determine the crosstalk protection duration such that there is a positive correlation between the magnitude of the pacing pulses and the crosstalk protection duration.

15. The medical device of claim 11, wherein the controller is configured to:
obtain information about a sense detection threshold of the sense circuit and determine the crosstalk protection duration based on the sense detection threshold of the sense circuit such that there is a negative correlation between the sense detection threshold of the sense circuit and the crosstalk protection duration; or
obtain information about a gain of the sense circuit and determine the crosstalk protection duration based on the gain of the sense circuit such that there is a positive correlation between the gain of the sense circuit and the crosstalk protection duration.

16. The medical device of claim 11, wherein the controller is configured to:
obtain information about the magnitude of the pacing pulses that the other leadless pacemaker is configured to deliver to the second cardiac chamber;
obtain information about the sensitivity of the sense circuit; and
determine the crosstalk protection duration based on both the magnitude of the pacing pulses that the other leadless pacemaker is configured to deliver to the second cardiac chamber and the sensitivity of the sense circuit.

17. A crosstalk protection method for use with a dual chamber leadless pacemaker (LP) system including a first leadless pacemaker (LP1) and a second leadless pacemaker (LP2), wherein the LP1 is configured to be implanted in or on a first cardiac chamber of a patient's heart and to deliver pacing pulses to the first cardiac chamber and the LP2 is configured to be implanted in or on a second cardiac chamber of the patient's heart and to deliver pacing pulses to the second cardiac chamber, the method comprising:
obtaining information about one or more of
a magnitude of the pacing pulses that the LP2 is configured to deliver to the second cardiac chamber, or
a sensitivity of a sense circuit of the LP1 that is configured to be used by the LP1 to detect intrinsic depolarizations of the first cardiac chamber;
determining a crosstalk protection duration based on at least some of the information;
the LP1 monitoring for and detecting possible crosstalk that may be caused by the LP2 delivering one of the pacing pulses to the second cardiac chamber; and
the LP1, in response to detecting the possible crosstalk that may be caused by the LP2 delivering one of the pacing pulses to the second cardiac chamber, initiating providing crosstalk protection for the crosstalk protection duration.

18. The method of claim 17, wherein:
the obtaining information comprises obtaining information about the magnitude of the pacing pulses that the LP2 is configured to deliver to the second cardiac chamber by obtaining information about at least one of a pulse amplitude or a pulse width of the pacing pulses that the LP2 is configured to deliver to the second cardiac chamber; and
the determining the crosstalk protection duration is based on the magnitude of the pacing pulses such that there is a positive correlation between the magnitude of the pacing pulses and the crosstalk protection duration.

19. The method of claim 17, wherein the obtaining information comprises obtaining information about the sensitivity of the sense circuit of the LP1, and wherein:
the sensitivity of the sense circuit of the LP1 is specified by a sense detection threshold of the sense circuit of the LP1 that is configured to be used by the LP1 to detect intrinsic depolarizations of the first cardiac chamber and the determining the crosstalk protection duration is based on the sense detection threshold of the sense circuit such that there is a negative correlation between the sense detection threshold of the sense circuit and the crosstalk protection duration; or
the sensitivity of the sense circuit of the LP1 is specified by a gain of the sense circuit of the LP1 that is configured to be used by the LP1 to detect intrinsic depolarizations of the first cardiac chamber and the determining the crosstalk protection duration is based on the gain of the sense circuit such that there is a positive correlation between the gain of the sense circuit and the crosstalk protection duration.

20. The method of claim 17, wherein:
the obtaining information comprises obtaining information about the magnitude of the pacing pulses that the LP2 is configured to deliver to the second cardiac chamber and obtaining information about the sensitivity of the sense circuit of the LP1 that is configured to be used by the LP1 to detect intrinsic depolarizations of the first cardiac chamber; and
the determining the crosstalk protection duration is based on the magnitude of the pacing pulses and the sensitivity of the sense circuit.

21. The method of claim 17, wherein the determining the crosstalk protection duration is performed by a controller of the LP1.

22. The method of claim 17, wherein:
the obtaining the information and the determining the crosstalk protection duration are performed by a non-implantable programmer; and
the method further comprises the non-implantable programmer programming the crosstalk protection duration into a memory or one or more registers of the LP1, so that the crosstalk protection duration is available for use by the LP1 when possible crosstalk that may be caused by the LP2 delivering one of the pacing pulses to the second cardiac chamber is detected by the LP1.

23. The method of claim 17, wherein the providing crosstalk protection for the crosstalk protection duration comprises at least one of the following:
blanking the sense circuit of the LP1 for the crosstalk protection duration;
ignoring any possible intrinsic depolarization detected using the sense circuit of the LP1 during the crosstalk protection duration;
disabling the sense circuit of the LP1 for the crosstalk protection duration;
ignoring any interrupts produced in response to the sense circuit being used to detect a possible intrinsic depolarization during the crosstalk protection duration; or
disabling generating of interrupts that may be produced in response to the sense circuit detecting a possible intrinsic depolarization during the crosstalk protection duration.

24. The method of claim 17, further comprising:
the LP1 determining that the possible crosstalk that was detected is part of a valid message transmitted by another device and in response thereto, terminating the crosstalk protection for a remainder of the crosstalk protection duration.
